(19) United States
(12) Patent Application Publication
(10) Pub. No.: US 2002/0157142 A1
(43) Pub. Date: Oct. 24, 2002

(54) Herbicide target genes and methods

(76) Inventors: Lyn Wegrich Glover, San Jose, CA (US); Gregory J. Budziszewski, Durham, NC (US); Qing Zhou, Raleigh, NC (US); Joshua Z. Levin, Raleigh, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 09/741,233

(22) Filed: Dec. 18, 2000

(51) Int. Cl. C12N 15/82; A01H 5/00; C12N 15/29

(52) U.S. Cl. 800/300; 536/23.6

(57) ABSTRACT

The invention relates to genes isolated from Arabidopsis that code for proteins essential for seedling growth. The invention also includes the methods of using these proteins to discover new herbicides, based on the essentiality of these genes for normal growth and development. The invention can also be used in a screening assay to identify inhibitors that are potential herbicides. The invention is also applied to the development of herbicide tolerant plants, plant tissues, plant seeds, and plant cells.

This application claims the benefit of U.S. Provisional Application No. ______ (Glover et al.) [docket no. PB/5-31127P1], filed on Dec. 16, 1999. The disclosure of this priority document is hereby expressly incorporated by reference in its entirety in the instant disclosure.

### FIELD OF THE INVENTION

The invention relates to genes isolated from Arabidopsis that code for proteins essential for seedling growth. The invention also includes the methods of using these proteins as herbicide targets, based on the essentiality of the genes for normal growth and development. The invention is also useful as a screening assay to identify inhibitors that are potential herbicides. The invention may also be applied to the development of herbicide tolerant plants, plant tissues, plant seeds, and plant cells.

### BACKGROUND OF THE INVENTION

The use of herbicides to control undesirable vegetation such as weeds in crop fields has become almost a universal practice. The herbicide market exceeds 15 billion dollars annually. Despite this extensive use, weed control remains a significant and costly problem for farmers.

Effective use of herbicides requires sound management. For instance, the time and method of application and stage of weed plant development are critical to getting good weed control with herbicides. Since various weed species are resistant to herbicides, the production of effective new herbicides becomes increasingly important. Novel herbicides can now be discovered using high-throughput screens that implement recombinant DNA technology. Metabolic enzymes found to be essential to plant growth and development can be recombinantly produced through standard molecular biological techniques and utilized as herbicide targets in screens for novel inhibitors of the enzyme activity. The novel inhibitors discovered through such screens may then be used as herbicides to control undesirable vegetation.

Herbicides that exhibit greater potency, broader weed spectrum, and more rapid degradation in soil can also, unfortunately, have greater crop phytotoxicity. One solution applied to this problem has been to develop crops that are resistant or tolerant to herbicides. Crop hybrids or varieties tolerant to the herbicides allow for the use of the herbicides to kill weeds without attendant risk of damage to the crop. Development of tolerance can allow application of a herbicide to a crop where its use was previously precluded or limited (e.g. to pre-emergence use) due to sensitivity of the crop to the herbicide. For example, U.S. Pat. No. 4,761,373 to Anderson et al. is directed to plants resistant to various imidazolinone or sulfonamide herbicides. An altered acetohydroxyacid synthase (AHAS) enzyme confers the resistance. U.S. Pat. No. 4,975,374 to Goodman et al. relates to plant cells and plants containing a gene encoding a mutant glutamine synthetase (GS) resistant to inhibition by herbicides that were known to inhibit GS, e.g. phosphinothricin and methionine sulfoximine. U.S. Pat. No. 5,013,659 to Bedbrook et al. is directed to plants expressing a mutant acetolactate synthase that renders the plants resistant to inhibition by sulfonylurea herbicides. U.S. Pat. No. 5,162,602 to Somers et al. discloses plants tolerant to inhibition by cyclohexanedione and aryloxyphenoxypropanoic acid herbicides. The tolerance is conferred by an altered acetyl coenzyme A carboxylase (ACCase).

Notwithstanding the above described advancements, there remain persistent and ongoing problems with unwanted or detrimental vegetation growth (e.g. weeds). Furthermore, as the population continues to grow, there will be increasing food shortages. Therefore, there exists a long felt, yet unfulfilled need, to find new, effective, and economic herbicides.

### SUMMARY OF THE INVENTION

It is an object of the invention to provide effective and beneficial methods to identify novel herbicides. A feature of the invention is the identification of genes in Arabidopsis, herein referred to as the ET1158 gene, which encodes a protein with sequence similarity to a DNA binding protein, possibly involved in sucrose transport (Kuhn and Frommer (1995) Mol. Gen. Genetics, 247: 759-763); the GT6839 gene, which encodes a protein with no known function, but may play a role in chloroplast protein import (Settles and Martienssen (1998) Trends in Cell Biology, 8: 494-501); and the ET5262 gene, which encodes a protein with sequence similarity to beta-1,3-glucanases (del Campillo, E (1999) Curr. Top. Dev. Biol. 46: 39-61). An important and unexpected feature of the invention is the discovery that each of these genes is essential for seedling growth and development. An advantage of the present invention is that the newly discovered essential genes containing novel herbicidal modes of action enable one skilled in the art to easily and rapidly identify novel herbicides.

One object of the present invention is to provide essential genes in plants for assay development for inhibitory compounds with herbicidal activity. Genetic results show that when either the ET1158, GT6839, or ET5262 genes are mutated in Arabidopsis, the resulting phenotype is seedling lethal in the homozygous state. This suggests a critical role for the gene products encoded by each of these genes.

Using Ac/Ds transposon mutagenesis, the inventors of the present invention have demonstrated that the activity encoded by the Arabidopsis ET1158, GT6839, or ET5262 genes (herein referred to as ET1158, GT6839, or ET5262 activity) is essential in Arabidopsis seedlings. This implies that chemicals that inhibit the function of any one of these proteins in plants are likely to have detrimental effects on plants and are potentially good herbicide candidates. The present invention therefore provides methods of using a purified protein encoded by any one of the gene sequences described below to identify inhibitors thereof, which can then be used as herbicides to suppress the growth of undesirable vegetation, e.g. in fields where crops are grown, particularly agronomically important crops such as maize and other cereal crops such as wheat, oats, rye, sorghum, rice, barley, millet, turf and forage grasses, and the like, as well as cotton, sugar cane, sugar beet, oilseed rape, and soybeans.

The present invention discloses a nucleotide sequence derived from Arabidopsis, designated the ET1158 gene. The nucleotide sequence of the cDNA clone is set forth in SEQ ID NO:1, and the corresponding amino acid sequence is set forth in SEQ ID NO:2. The nucleotide sequence of the genomic DNA sequence is set forth in SEQ ID NO:19. Also, the present invention discloses a nucleotide sequence derived from Arabidopsis, designated the GT6839 gene. The nucleotide sequence of the cDNA clone is set forth in SEQ ID NO:3, and the corresponding amino acid sequence is set forth in SEQ ID NO:4. The nucleotide sequence of the genomic DNA sequence is set forth in SEQ ID NO:20. Furthermore, the present invention discloses a nucleotide sequence derived from Arabidopsis, designated the ET5262 gene. The nucleotide sequence of the cDNA clone is set forth in SEQ ID NO:5, and the corresponding amino acid sequence is set forth in SEQ ID NO:6. The nucleotide sequence of the genomic DNA sequence is set forth in SEQ ID NO:21. The present invention also includes nucleotide sequences substantially similar to those set forth in SEQ ID NO:1, SEQ ID NO:3, and SEQ ID NO:5. The present invention also encompasses plant proteins whose amino acid sequence are substantially similar to the amino acid sequences set forth in SEQ ID NO:2, SEQ ID NO:4, and SEQ ID NO:6. Such proteins can be used in a screening assay to identify inhibitors that are potential herbicides.

In a preferred embodiment, the present invention relates to a method for identifying chemicals having the ability to inhibit ET1158, GT6839, or ET5262 activity in plants preferably comprising the steps of: a) obtaining transgenic plants, plant tissue, plant seeds or plant cells, preferably stably transformed, comprising a non-native nucleotide sequence encoding an enzyme having ET1158, GT6839, or ET5262 activity, respectively, and capable of overexpressing an enzymatically active ET1158, GT6839, or ET5262 gene product (either full length or truncated but still active), respectively; b) applying a chemical to the transgenic plants, plant cells, tissues or parts and to the isogenic non-transformed plants, plant cells, tissues or parts; c) determining the growth or viability of the transgenic and non-transformed plants, plant cells, tissues after application of the chemical; d) comparing the growth or viability of the transgenic and non-transformed plants, plant cells, tissues after application of the chemical; and e) selecting chemicals that suppress the viability or growth of the non-transgenic plants, plant cells, tissues or parts, without significantly suppressing the growth of the viability or growth of the isogenic transgenic plants, plant cells, tissues or parts. In a preferred embodiment, the enzyme having ET1158, GT6839, or ET5262 activity is encoded by a nucleotide sequence derived from a plant, preferably a monocotyledonous or a dicotyledonous plant, preferably a dicotyledonous plant, preferably Arabidopsis thaliana, desirably identical or substantially similar to the nucleotide sequence set forth in SEQ ID NO:1, SEQ ID NO:3, or SEQ ID NO:5, respectively. In another embodiment, the enzyme having ET1158, GT6839, or ET5262 activity is encoded by a nucleotide sequence capable of encoding the amino acid sequence of SEQ ID NO:2, SEQ ID NO:4, or SEQ ID NO:6, respectively. In yet another embodiment, the enzyme having ET1158, GT6839, or ET5262 activity has an amino acid sequence identical or substantially similar to the amino acid sequence set forth in SEQ ID NO:2, SEQ ID NO:4, or SEQ ID NO:6, respectively.

The present invention further embodies plants, plant tissues, plant seeds, and plant cells that have modified ET1158, GT6839, or ET5262 activity and that are therefore tolerant to inhibition by a herbicide at levels normally inhibitory to naturally occurring ET1158, GT6839, or ET5262 activity, respectively. Herbicide tolerant plants encompassed by the invention include those that would otherwise be potential targets for normally inhibiting herbicides, particularly the agronomically important crops mentioned above. According to this embodiment, plants, plant tissue, plant seeds, or plant cells are transformed, preferably stably transformed, with a recombinant DNA molecule comprising a suitable promoter functional in plants operatively linked to a nucleotide coding sequence that encodes a modified ET1158, GT6839, or ET5262 gene that is tolerant to inhibition by a herbicide at a concentration that would normally inhibit the activity of wild-type, unmodified ET1158, GT6839, or ET5626 gene product, respectively. Modified ET1158, GT6839, or ET5262 activity may also be conferred upon a plant by increasing expression of wild-type herbicide-sensitive ET1158, GT6839, or ET5262 protein by providing multiple copies of wild-type ET1158, GT6839, or ET5262 genes, respectively, to the plant or by overexpression of wild-type ET1158, GT6839, or ET5262 genes, respectively, under control of a stronger-than-wild-type promoter. The transgenic plants, plant tissue, plant seeds, or plant cells thus created are then selected by conventional selection techniques, whereby herbicide tolerant lines are isolated, characterized, and developed. Alternately, random or site-specific mutagenesis may be used to generate herbicide tolerant lines.

Therefore, the present invention provides a plant, plant cell, plant seed, or plant tissue transformed with a DNA molecule comprising a nucleotide sequence isolated from a plant that encodes an enzyme having ET1158, GT6839, or ET5262 activity, wherein the DNA expresses the ET1158, GT6839, or ET5262 activity, respectively, and wherein the DNA molecule confers upon the plant, plant cell, plant seed, or plant tissue tolerance to a herbicide in amounts that normally inhibits naturally occurring ET1158, GT6839, or ET5262 activity, respectively. According to one example of this embodiment, the enzyme having ET1158, GT6839, or ET5262 activity is encoded by a nucleotide sequence identical or substantially similar to the nucleotide sequence set forth in SEQ ID NO:1, SEQ ID NO:3 or SEQ ID NO:5, respectively, or has an amino acid sequence identical or substantially similar to the amino acid sequence set forth in SEQ ID NO:2, SEQ ID NO:4, or SEQ ID NO:6, respectively.

The invention also provides a method for suppressing the growth of a plant comprising the step of applying to the plant a chemical that inhibits the naturally occurring ET1158, GT6839, or ET5262 activity in the plant. In a related aspect, the present invention is directed to a method for selectively suppressing the growth of undesired vegetation in a field containing a crop of planted crop seeds or plants, comprising the steps of: (a) optionally planting herbicide tolerant crops or crop seeds, which are plants or plant seeds that are tolerant to a herbicide that inhibits the naturally occurring ET1158, GT6839, or ET5262 activity; and (b) applying to the herbicide tolerant crops or crop seeds and the undesired vegetation in the field a herbicide in amounts that inhibit naturally occurring ET1158, GT6839, or ET5262 activity, respectively, wherein the herbicide suppresses the growth of the weeds without significantly suppressing the growth of the crops.

The invention thus provides:

An isolated DNA molecule comprising a nucleotide sequence substantially similar to SEQ ID NO:1, SEQ ID NO:3, or SEQ ID NO:5. In a preferred embodiment, the nucleotide sequence encodes an amino acid sequence substantially similar to SEQ ID NO:2, SEQ ID NO:4, or SEQ ID NO:6. In another preferred embodiment, the nucleotide sequence is SEQ ID NO:1, SEQ ID NO:3, or SEQ ID NO:5. In yet another preferred embodiment, the nucleotide sequence encodes the amino acid sequence of SEQ ID NO:2, SEQ ID NO:4, or SEQ ID NO:6. Preferably, the nucleotide sequence is a plant nucleotide sequence, which preferably encodes a polypeptide having ET1158, GT6839, or ET5262 activity, respectively.

The invention further provides:

A polypeptide comprising an amino acid sequence encoded by a nucleotide sequence substantially similar to SEQ ID NO:1, SEQ ID NO:3, or SEQ ID NO:5. Preferably, the amino acid sequence is encoded by SEQ ID NO:1, SEQ ID NO:3, or SEQ ID NO:5. Preferably, the polypeptide comprises an amino acid sequence substantially similar to SEQ ID NO:2, SEQ ID NO:4, or SEQ ID NO:6, respectively. Preferably the amino acid sequence is SEQ ID NO:2, SEQ ID NO:4, or SEQ ID NO:6. The amino acid sequence preferably has ET1158, GT6839, or ET5262 activity, respectively. In another preferred embodiment, the amino acid sequence comprises at least 20 consecutive amino acid residues of the amino acid sequence encoded by SEQ ID NO:1, SEQ ID NO:3, or SEQ ID NO:5, respectively. Or, alternatively, the amino acid sequence comprises at least 20 consecutive amino acid residues of the amino acid sequence of SEQ ID NO:2, SEQ ID NO:4, or SEQ ID NO:6, respectively.

The invention further provides:

An expression cassette comprising a promoter operatively linked to a DNA molecule according to the present invention, a recombinant vector comprising an expression cassette according to the present invention, wherein said vector is preferably capable of being stably transformed into a host cell, a host cell comprising a DNA molecule according to the present invention, wherein said DNA molecule is preferably expressible in the cell. The host cell is preferably selected from the group consisting of an insect cell, a yeast cell, a prokaryotic cell and a plant cell. The invention further provides a plant or seed comprising a plant cell of the present invention, wherein the plant or seed is preferably tolerant to an inhibitor of ET1158, GT6839, or ET5262 activity, respectively.

The invention further provides:

A process for making nucleotides sequences encoding gene products having altered ET1158, GT6839, or ET5262 activity, comprising: a) shuffling an unmodified nucleotide sequence of the present invention, b) expressing the resulting shuffled nucleotide sequences, and c) selecting for altered ET1158, GT6839, or ET5262 activity, respectively, as compared to the Etl 158, GT6839, or ET5262 activity, respectively, of the gene product of said unmodified nucleotide sequence.

In a preferred embodiment, the unmodified nucleotide sequence is identical or substantially similar to SEQ ID NO:1, SEQ ID NO:3, or SEQ ID NO:5, respectively, or a homolog thereof. The present invention further provides a DNA molecule comprising a shuffled nucleotide sequence obtainable by the process described above, a DNA molecule comprising a shuffled nucleotide sequence produced by the process described above. Preferably, a shuffled nucleotide sequence obtained by the process described above has enhanced tolerance to an inhibitor of ET1158, GT6839, or ET5262 activity, respectively. The invention further provides an expression cassette comprising a promoter operatively linked to a DNA molecule comprising a shuffled nucleotide sequence a recombinant vector comprising such an expression cassette, wherein said vector is preferably capable of being stably transformed into a host cell, a host cell comprising such an expression cassette, wherein said nucleotide sequence is preferably expressible in said cell. A preferred host cell is selected from the group consisting of an insect cell, a yeast cell, a prokaryotic cell and a plant cell. The invention further provides a plant or seed comprising such plant cell, wherein the plant is preferably tolerant to an inhibitor of ET1158, GT6839, or ET5262 activity, respectively.

The invention further provides:

A method for selecting compounds that interact with the protein encoded by SEQ ID NO:1 SEQ ID NO:3, or SEQ ID NO:5, respectively, comprising: a) expressing a DNA molecule comprising SEQ ID NO:1, SEQ ID NO:3, or SEQ ID NO:5, respectively, or a sequence substantially similar to SEQ ID NO:1, SEQ ID NO:3, or SEQ ID NO:5, respectively, or a homolog thereof, to generate the corresponding protein, b) testing a compound suspected of having the ability to interact with the protein expressed in step (a), and c) selecting compounds that interact with the protein in step (b).

The invention further provides:

A process of identifying an inhibitor of ET1158, GT6839, or ET5262 activity, respectively, comprising: a) introducing a DNA molecule comprising a nucleotide sequence of SEQ ID NO:1, SEQ ID NO:3, or SEQ ID NO:5, respectively, and having ET1158, GT6839, or ET5262 activity, respectively, or nucleotide sequences substantially similar thereto, or a homolog thereof, into a plant cell, such that said sequence is functionally expressible at levels that are higher than wild-type expression levels, b) combining said plant cell with a compound to be tested for the ability to inhibit the ET1158, GT6839, or ET5262 activity, respectively, under conditions conducive to such inhibition, c) measuring plant cell growth under the conditions of step (b), d) comparing the growth of said plant cell with the growth of a plant cell having unaltered ET1158, GT6839, or ET5262 activity, respectively, under identical conditions, and e) selecting said compound that inhibits plant cell growth in step (d).

The invention further comprises a compound having herbicidal activity identifiable according to the process described immediately above.

The invention further comprises:

A process of identifying compounds having herbicidal activity comprising: a) combining a protein of the present invention and a compound to be tested for the ability to interact with said protein, under conditions conducive to interaction, b) selecting a compound identified in step (a) that is capable of interacting with said protein, c) applying identified compound in step (b) to a plant to test for herbicidal activity, and d) selecting compounds having herbicidal activity.

The invention further comprises a compound having herbicidal activity identifiable according to the process described immediately above.

The invention further comprises:

A method for suppressing the growth of a plant comprising, applying to said plant a compound that inhibits the activity of a polypeptide of the present invention in an amount sufficient to suppress the growth of said plant.

The invention further comprises:

A method for recombinantly expressing a protein having ET1158, GT6839, or ET5262 activity comprising introducing a nucleotide sequence encoding a protein having one of the above activities into a host cell and expressing the nucleotide sequence in the host cell. Preferably, the protein is substantially similar to SEQ ID NO:2, SEQ ID NO:4, or SEQ ID NO:6. Preferably, the nucleotide sequence is substantially similar to SEQ ID NO:1, SEQ ID NO:3, or SEQ ID NO:5. A preferred host cell is selected from the group consisting of an insect cell, a yeast cell, a prokaryotic cell and a plant cell. A preferred prokaryotic cell is a bacterial cell, e.g. E. coli.

Other objects and advantages of the present invention will become apparent to those skilled in the art from a study of the following description of the invention and non-limiting examples.

### Definitions

For clarity, certain terms used in the specification are defined and presented as follows

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

DNA shuffling: DNA shuffling is a method to rapidly, easily and efficiently introduce mutations or rearrangements, preferably randomly, in a DNA molecule or to generate exchanges of DNA sequences between two or more DNA molecules, preferably randomly. The DNA molecule resulting from DNA shuffling is a shuffled DNA molecule that is a non-naturally occurring DNA molecule derived from at least one template DNA molecule. The shuffled DNA encodes an enzyme modified with respect to the enzyme encoded by the template DNA, and preferably has an altered biological activity with respect to the enzyme encoded by the template DNA.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

“ET1158 Gene” as used herein refers to a DNA molecule comprising a nucleotide sequence encoding SEQ ID NO:2, or a nucleotide sequence substantially similar thereto. Preferably, the nucleotide sequence is set forth in SEQ ID NO:1 or is substantially similar to SEQ ID NO:1.

“GT6839 Gene” as used herein refers to a DNA molecule comprising a nucleotide sequence encoding SEQ ID NO:4, or a nucleotide sequence substantially similar thereto. Preferably, the nucleotide sequence is set forth in SEQ ID NO:3 or is substantially similar to SEQ ID NO:3.

“ET5262 Gene” as used herein refers to a DNA molecule comprising a nucleotide sequence encoding SEQ ID NO:6, or a nucleotide sequence substantially similar thereto. Preferably, the nucleotide sequence is set forth in SEQ ID NO:5 or is substantially similar to SEQ ID NO:5.

Herbicide: a chemical substance used to kill or suppress the growth of plants, plant cells, plant seeds, or plant tissues.

Heterologous DNA Sequence: a DNA sequence not naturally associated with a host cell into which it is introduced, including non-naturally occurring multiple copies of a naturally occurring DNA sequence; and genetic constructs wherein an otherwise homologous DNA sequence is operatively linked to a non-native sequence.

Homologous DNA Sequence: a DNA sequence naturally associated with a host cell into which it is introduced.

Inhibitor: a chemical substance that causes abnormal growth, e.g., by inactivating the enzymatic activity of a protein such as a biosynthetic enzyme, receptor, signal transduction protein, structural gene product, or transport protein that is essential to the growth or survival of the plant. In the context of the instant invention, an inhibitor is a chemical substance that alters the enzymatic activity encoded by the ET1158, GT6839, or ET5262 gene from a plant. More generally, an inhibitor causes abnormal growth of a host cell by interacting with the gene product encoded by the ET1158, GT6839, or ET5262 gene.

Isogenic: plants which are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein which is normally targeted to a cellular organelle, such as a chloroplast, and from which the transit peptide has been removed.

Minimal Promoter: promoter elements, particularly a TATA element, that are inactive or that have greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Pre-protein: protein which is normally targeted to a cellular organelle, such as a chloroplast, and still comprising its transit peptide.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

In its broadest sense, the term “substantially similar”, when used herein with respect to a nucleotide sequence, means a nucleotide sequence corresponding to a reference nucleotide sequence, wherein the corresponding sequence encodes a polypeptide having substantially the same structure and function as the polypeptide encoded by the reference nucleotide sequence. Desirably the substantially similar nucleotide sequence encodes the polypeptide encoded by the reference nucleotide sequence. The term “substantially similar” is specifically intended to include nucleotide sequences wherein the sequence has been modified to optimize expression in particular cells. In the context of the “ET1158 gene”, “substantially similar” refers to nucleotide sequences that encode a protein at least 57% identical, more preferably at least 65% identical, still more preferably at least 75% identical, still more preferably at least 85% identical, still more preferably at least 95% identical, yet still more preferably at least 99% identical to SEQ ID NO:2; in the context of the “GT6839 gene”, “substantially similar” refers to nucleotide sequences that encode a protein at least 59% identical, more preferably at least 65% identical, still more preferably at least 75% identical, still more preferably at least 85% identical, still more preferably at least 95% identical, yet still more preferably at least 99% identical to SEQ ID NO:4; in the context of the “ET5262 gene”, “substantially similar” refers to nucleotide sequences that encode a protein at least 43% identical, more preferably at least 55% identical, more preferably at least 65% identical, still more preferably at least 75% identical, still more preferably at least 85% identical, still more preferably at least 95% identical, yet still more preferably at least 99% identical to SEQ ID NO:6, wherein said protein sequence comparisons are conducted using GAP analysis as described below. A nucleotide sequence “substantially similar” to the reference nucleotide sequence hybridizes to the reference nucleotide sequence in 7% sodium dodecyl sulfate (SDS), 0.5 M NaPO₄, 1 mM EDTA at 50° C. with washing in 2× SSC, 0.1% SDS at 50° C., more desirably in 7% sodium dodecyl sulfate (SDS), 0.5 M NaPO₄, 1 mM EDTA at 50° C. with washing in I× SSC, 0.1% SDS at 50° C., more desirably still in 7% sodium dodecyl sulfate (SDS), 0.5 M NaPO₄, 1 mM EDTA at 50° C. with washing in 0.5× SSC, 0.1% SDS at 50° C., preferably in 7% sodium dodecyl sulfate (SDS), 0.5 M NaPO₄, 1 mM EDTA at 50° C. with washing in 0.1× SSC, 0.1% SDS at 50° C., more preferably in 7% sodium dodecyl sulfate (SDS), 0.5 M NaPO₄, 1 mM EDTA at 50° C. with washing in 0.1× SSC, 0.1% SDS at 65° C.

“Homologs of the ET1158 gene” include nucleotide sequences that encode an amino acid sequence that is at least 20% identical to SEQ ID NO:2, more preferably at least 30% identical, still more preferably at least 40% identical, still more preferably at least 48% identical, yet still more preferably at least 56% identical, still more preferably at least 65% identical, yet still more preferably at least 75% identical to SEQ ID NO:2, as measured, using the GAP parameters described below, wherein the amino acid sequence encoded by the homolog has the biological activity of the ET1158 protein.

“Homologs of the GT6839 gene” include nucleotide sequences that encode an amino acid sequence that is at least 30% identical to SEQ ID NO:4, more preferably at least 40% identical, still more preferably at least 50% identical, still more preferably at least 60% identical, yet still more preferably at least 80% identical to SEQ ID NO:4, as measured, using the GAP parameters described below, wherein the amino acid sequence encoded by the homolog has the biological activity of the GT6839 protein.

“Homologs of the ET5262 gene” include nucleotide sequences that encode an amino acid sequence that is at least 20% identical to SEQ ID NO:6, more preferably at least 25% identical, still more preferably at least 30% identical, still more preferably at least 40% identical, yet still more preferably at least 60% identical to SEQ ID NO:6, as measured, using the GAP parameters described below, wherein the amino acid sequence encoded by the homolog has the biological activity of the ET5262 protein.

The term “substantially similar”, when used herein with respect to a protein, means a protein corresponding to a reference protein, wherein the protein has substantially the same structure and function as the reference protein, e.g. where only changes in amino acids sequence not affecting the polypeptide function occur. When used in the context of the “ET1158 gene”, the percentage of identity between the substantially similar protein or amino acid sequence and the reference protein or amino acid sequence (in this case SEQ ID NO:2) is at least 57%, more preferably at least 65%, still more preferably at least 75%, still more preferably at least 85%, still more preferably at least 95%, yet still more preferably at least 99%, as determined using default GAP analysis parameters with the University of Wisconsin GCG, SEQWEB application of GAP, based on the algorithm of Needleman and Wunsch (Needleman and Wunsch (1970) J Mol. Biol. 48: 443-453). In the context of the “GT6839 gene”, the percentage of identity between the substantially similar protein or amino acid sequence and the reference protein or amino acid sequence (in this case SEQ ID NO:4) is at least 59%, more preferably at least 65%, still more preferably at least 75%, still more preferably at least 85%, still more preferably at least 95%, yet still more preferably at least 99%. In the context of the “ET5262 gene”, the percentage of identity between the substantially similar protein or amino acid sequence and the reference protein or amino acid sequence (in this case SEQ ID NO:6) is at least 43%, more preferably at least 55%, more preferably at least 65%, still more preferably at least 75%, still more preferably at least 85%, still more preferably at least 95%, yet still more preferably at least 99%.

As used herein the term “ET1158 protein” refers to an amino acid sequence encoded by a DNA molecule comprising a nucleotide sequence substantially similar to SEQ ID NO:1. “Homologs of the ET1158 protein” are amino acid sequences that are at least 20% identical to SEQ ID NO:2, more preferably at least 30% identical, still more preferably at least 40% identical, still more preferably at least 48% identical, yet still more preferably at least 56% identical, still more preferably at least 65% identical, yet still more preferably at least 75% identical to SEQ ID NO:2, as measured using the GAP parameters described above, wherein the homologs of the ET1158 protein have the biological activity of the ET1158 protein.

As used herein the term “GT6839 protein” refers to an amino acid sequence encoded by a DNA molecule comprising a nucleotide sequence substantially similar to SEQ ID NO:3. “Homologs of the GT6839 protein” are amino acid sequences that are at least 30% identical, more preferably at least 40% identical, still more preferably at least 50% identical, still more preferably at least 60% identical, yet still more preferably at least 80% identical to SEQ ID NO:4, as measured using the GAP parameters described above, wherein the homologs of the GT6839 protein have the biological activity of the GT6839 protein.

As used herein the term “ET5262 protein” refers to an amino acid sequence encoded by a DNA molecule comprising a nucleotide sequence substantially similar to SEQ ID NO:5. “Homologs of the ET5262 protein” are amino acid sequences that are at least at least 20% identical, more preferably at least 25% identical, still more preferably at least 30% identical, still more preferably at least 40% identical, yet still more preferably at least 60% identical to SEQ ID NO:6, as measured using the GAP parameters described above, wherein the homologs of the ET5262 protein have the biological activity of the ET5262 protein.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Tolerance: the ability to continue essentially normal growth or function (i.e. no more than 5% of herbicide tolerant plants show phytotoxicity) when exposed to an inhibitor or herbicide in an amount sufficient to suppress the normal growth or function of native, unmodified plants.

Transformation: a process for introducing heterologous DNA into a cell, tissue, or plant. Transformed cells, tissues, or plants are understood to encompass not only the end product of a transformation process, but also transgenic progeny thereof.

Transgenic: stably transformed with a recombinant DNA molecule that preferably comprises a suitable promoter operatively linked to a DNA sequence of interest.

### BRIEF DESCRIPTION OF THE SEQUENCES IN THE SEQUENCE LISTING

SEQ ID NO:1 cDNA coding sequence for the Arabidopsis ET1158 gene

SEQ ID NO:2 amino acid sequence encoded by the Arabidopsis ET1158 nucleotide sequence shown in SEQ ID NO:1

SEQ ID NO:3 CDNA coding sequence of the Arabidopsis GT6839 gene

SEQ ID NO:4 amino acid sequence encoded by the Arabidopsis GT6839 nucleotide sequence shown in SEQ ID NO:3

SEQ ID NO:5 cDNA coding sequence for the Arabidopsis ET5262 gene

SEQ ID NO:6 amino acid sequence encoded by the Arabidopsis ET5262 nucleotide sequence shown in SEQ ID NO:5

SEQ ID NO:7 oligonucleotide LWAD1

SEQ ID NO:8 oligonucleotide CA51

SEQ ID NO:9 oligonucleotide CA52

SEQ ID NO:10 oligonucleotide CA53

SEQ ID NO:11 oligonucleotide CA54

SEQ ID NO:12 oligonucleotide CA55

SEQ ID NO:13 oligonucleotide 5A

SEQ ID NO:14 oligonucleotide 5B

SEQ ID NO:15 oligonucleotide 5C

SEQ ID NO:16 oligonucleotide 3A

SEQ ID NO:17 oligonucleotide 3B

SEQ ID NO:18 oligonucleotide 3C

SEQ ID NO:19 genomic sequence of the Arabidopsis ET 1158 gene

SEQ ID NO:20 genomic sequence of the Arabidopsis GT6839 gene

SEQ ID NO:21 genomic sequence of the Arabidopsis ET5262 gene

### DETAILED DESCRIPTION OF THE INVENTION

I. Essentiality of the ET1158, GT6839, and ET6252 Genes inArabidopsis Demonstrated by Ac/Ds Transposon Mutagenesis

As shown in the examples below, the identification of a novel gene structure, as well as the essentiality of the ET1158, GT6839, or ET5262 genes for normal plant growth and development, have been demonstrated for the first time in Arabidopsis using Ac/Ds transposon mutagenesis. Having established the essentiality of ET1158, GT6839, and ET5262 functions in plants and having identified the genes encoding these essential activities, the inventors thereby provide an important and sought after tool for new herbicide development.

Arabidopsis insertional mutant lines segregating for seedling lethal mutations are identified as a first step in the identification of essential proteins. Ds transposon insertion lines were produced as described in Sundareson et al. (1995) Genes and Dev., 9:1797-1810), incorporated herein by reference. Starting with F3 or F4 seeds collected from single F2 or F3 kanamycin-resistant plants containing Ds insertions in their genomes (see FIG. 3 of Sundareson et al. (1995) Genes and Dev., 9:1797-1810), those lines segregating homozygous seedling lethal seedlings are identified. These lines are found by placing seeds onto minimal plant growth media, which contains the fungicides benomyl and maxim, and screening for inviable seedlings after 7 and 14 days in the light at room temperature. Inviable phenotypes include altered pigmentation or altered morphology. These phenotypes are observed either on plates directly or in soil following transplantation of seedlings.

When a line is identified as segregating a seedling lethal, it is determined if the resistance marker in the Ds transposon insertion co-segregates with the lethality (Errampalli et al. (1991) The Plant Cell, 3:149-157). Co-segregation analysis is done by placing the seeds on media containing the selective agent and scoring the seedlings for resistance or sensitivity to the agent. Examples of selective agents used are kanamycin, hygromycin, or phosphinothricin. About 35 resistant seedlings are transplanted to soil and their progeny are examined for the segregation of the seedling lethal. In the case in which the Ds transposon insertion disrupts an essential gene, there is co-segregation of the resistance phenotype and the seedling lethal phenotype in every plant. Therefore, in such a case, all resistant plants segregate seedling lethals in the next generation; this result indicates that each of the resistant plants is heterozygous for the DNA causing both phenotypes.

For the Arabidopsis lines showing co-segregation of the transposon-encoded resistance marker and the lethal phenotype, PCR-based molecular approaches such as, TAIL-PCR (Liu et al. (1995) The Plant Journal, 8:457-463; Liu and Whittier (1995), Genomics, 25: 674-681), vectorette PCR (Riley et al. (1990) Nucleic Acids Research, 18: 2887-2890)), or the GenomeWalker™ kit (CLONTECH Laboratories, Inc., Palo Alto, Calif.), may be used to directly amplify the plant DNA fragments flanking the transposon. Each of these techniques utilizes the known sequence of the transposon, and can be used to recover small (less than 5 KB) fragments directly adjacent to the insertion. PCR products are isolated and their DNA sequence is determined. The resulting sequences are analyzed for the presence of non-Ds transposon vector sequences. When such sequences are found, they are used to search DNA and protein databases using the BLAST and BLAST2 programs (Altschul et al. (1990) J Mol. Biol. 215: 403-410; Altschul et al (1997) Nucleic Acid Res. 25:3389-3402, both incorporated herein by reference). Additional genomic and cDNA sequences for each gene are identified by standard molecular biology procedures.

II. Sequence of the Arabidopsis ET1158 Gene

The Arabidopsis ET1158 gene is identified by isolating DNA flanking the Ds transposon border from the tagged seedling-lethal line #ET1158. A region of the Arabidopsis DNA flanking the Ds transposon border shows 100% identity to Arabidopsis genomic sequence (chromosome 2 of BAC F504, GenBank accession # AC005936). The inventors are the first to demonstrate that the ET1158 gene product is essential for normal growth and development in plants, as well as defining the function of the ET1158 gene through protein homology. The present invention discloses the cDNA coding nucleotide sequence of the Arabidopsis ET1158 gene as well as the amino acid sequence of the Arabidopsis ET1158 protein.

The present invention also encompasses an isolated amino acid sequence derived from a plant, wherein said amino acid sequence is identical or substantially similar to the amino acid sequence encoded by the nucleotide sequence set forth in SEQ ID NO:1, wherein said amino acid sequence has ET1158 activity. Using BLAST and BLAST2 programs with the default settings, the sequence of the ET1158 gene shows similarity to a DNA binding protein, possibly involved in sucrose transport, from fruit fly, Drosophila melanogaster (Genbank Accession # CAA78696), house mouse, Mus musculus (Genbank Accession # AAD45924), Chinese hamster, Cricetulus griseus (Genbank Accession # AAC53577), human, Homo sapiens (PIR Accession # S47068), potato, Solanum tuberosum (PIR Accession # S48856), maize, Zea mays (Genbank Accession # ACC18941), and Arabidopsis thaliana (Genbank Accession #'s AAD20087, AAC78253, AAD10684, AAC97277).

III. Sequence of the Arabidopsis GT6839 Gene

The Arabidopsis GT6839 gene is identified by isolating DNA flanking the Ds transposon border from the tagged seedling-lethal line #GT6839. A region of the Arabidopsis DNA flanking the Ds transposon border shows *100% identity to Arabidopsis genomic sequence (chromosome 2, clone IGF-F23H14, working draft sequence, Genbank accession number AC006837). The inventors are the first to demonstrate that the GT6839 gene product is essential for normal growth and development in plants, as well as defining the function of the GT6839 gene through protein homology. The present invention discloses the cDNA coding nucleotide sequence of the Arabidopsis GT6839 gene as well as the amino acid sequence of the Arabidopsis GT6839 protein.

The present invention also encompasses an isolated amino acid sequence derived from a plant, wherein said amino acid sequence is identical or substantially similar to the amino acid sequence encoded by the nucleotide sequence set forth in SEQ ID NO:3, wherein said amino acid sequence has GT6839 activity. Using BLAST and BLAST2 programs with the default settings, the sequence of the GT6839 gene shows similarity to other proteins which may play a role in chloroplast protein import, including Ycf43 (Synechococcus, Genbank accession number AAD26593), hypothetical protein 27.8 kd (Synechocystis, SWISS PROT accession number P54086), hypothetical protein 28.1 kd (Porhpyra purpurea, SWISS PROT accession number P51264), hypothetical chloroplast RF43 (Guillardia theta, Genbank accession number AAC35684), hypothetical protein 30.1 kd YCF43 (Odontella sinensis, SWISS PROT accession number P49538), E. coli TATC (SWISS PROT accession number P27857), and hypothetical protein (Haemophilus influenzae, SWISS PROT accession number P44560).

IV. Sequence of the Arabidopsis ET5262 Gene

The Arabidopsis ET5262 gene is identified by isolating DNA flanking the Ds transposon border from the tagged seedling-lethal line #ET5262. The inventors are the first to demonstrate that the ET5262 gene product is essential for normal growth and development in plants, as well as defining the function of the ET5262 gene through protein homology. The present invention discloses the cDNA coding nucleotide sequence of the Arabidopsis ET5262 gene as well as the amino acid sequence of the Arabidopsis ET5262 protein.

The present invention also encompasses an isolated amino acid sequence derived from a plant, wherein said amino acid sequence is identical or substantially similar to the amino acid sequence encoded by the nucleotide sequence set forth in SEQ ID NO:5, wherein said amino acid sequence has ET5262 activity. Using BLAST and BLAST2 programs with the default settings, the sequence of the ET5262 gene shows similarity to other putative proteins similar to 1-3 beta endoglucanases from Arabidopsis thaliana (Genbank accession numbers CAB41118 and AAB97119), as well as other plant species, including wheat (Triticum aestivum, SWISS PROT accession number P52409) and rape (Brassica napus, PIR accession number S31612).

V. Recombinant Production of ET1158, GT6839, or ET5262 Activities and Uses Thereof

For recombinant production of ET1158, GT6839, or ET5262 activities in a host organism, a nucleotide sequence encoding a protein having ET1158, GT6839, or ET5262 activity, respectively, is inserted into an expression cassette designed for the chosen host and introduced into the host where it is recombinantly produced. For example, SEQ ID NO:1, nucleotide sequences substantially similar to SEQ ID NO:1, or homologs of the ET1158 gene are used for the recombinant production of a protein having ET1158 activity. For example, SEQ ID NO:3, nucleotide sequences substantially similar to SEQ ID NO:3, or homologs of the GT6839 gene are used for the recombinant production of a protein having GT6839 activity. For example, SEQ ID NO:5, nucleotide sequences substantially similar to SEQ ID NO:5, or homologs of the ET5262 gene are be used for the recombinant production of a protein having ET5262 activity. The choice of specific regulatory sequences such as promoter, signal sequence, 5′ and 3′ untranslated sequences, and enhancer appropriate for the chosen host is within the level of skill of the routineer in the art. The resultant molecule, containing the individual elements operably linked in proper reading frame, may be inserted into a vector capable of being transformed into the host cell. Suitable expression vectors and methods for recombinant production of proteins are well known for host organisms such as E. coli, yeast, and insect cells (see, e.g., Luckow and Summers, Bio/Technol. 6: 47 (1988), and baculovirus expression vectors, e.g., those derived from the genome of Autographica californica nuclear polyhedrosis virus (AcMNPV). A preferred baculovirus/insect system is pAcHLT (Pharmingen, San Diego, Calif.) used to transfect Spodoptera frugiperda Sf9 cells (ATCC) in the presence of linear Autographa californica baculovirus DNA (Pharmingen, San Diego, Calif.). The resulting virus is used to infect HighFive Tricoplusia ni cells (Invitrogen, La Jolla, Calif.).

In a preferred embodiment, the nucleotide sequence encoding a protein having ET1158, GT6839, or ET5262 activity is derived from an eukaryote, such as a mammal, a fly or a yeast, but is preferably derived from a plant, preferably a monocotyledonous or a dicotyledonous plant. In a further preferred embodiment, the nucleotide sequence is identical or substantially similar to the nucleotide sequence set forth in SEQ ID NO:1, or encodes a protein having ET1158 activity, whose amino acid sequence is identical or substantially similar to the amino acid sequence set forth in SEQ ID NO:2. In a further preferred embodiment, the nucleotide sequence is identical or substantially similar to the nucleotide sequence set forth in SEQ ID NO:3, or encodes a protein having GT6839 activity, whose amino acid sequence is identical or substantially similar to the amino acid sequence set forth in SEQ ID NO:4. In a further preferred embodiment, the nucleotide sequence is identical or substantially similar to the nucleotide sequence set forth in SEQ ID NO:5, or encodes a protein having ET5262 activity, whose amino acid sequence is identical or substantially similar to the amino acid sequence set forth in SEQ ID NO:6. In another preferred embodiment, the nucleotide sequence encoding a protein having ET1158, GT6839, or ET5262 activity, respectively, is derived from a prokaryote. Recombinantly produced protein having ET1158, GT6839, or ET5262 activity is isolated and purified using a variety of standard techniques. The actual techniques that may be used will vary depending upon the host organism used, whether the protein is designed for secretion, and other such factors familiar to the skilled artisan (see, e.g. chapter 16 of Ausubel, F. et al., “Current Protocols in Molecular Biology”, pub. by John Wiley & Sons, Inc. (1994).

Assays Utilizing the ET1158, GT6839, or ET5262 Proteins

Recombinantly produced proteins having ET1158, GT6839, or ET5262 activity are useful for a variety of purposes. For example, they can be used in in vitro assays to screen known herbicidal chemicals whose target has not been identified to determine if they inhibit ET1158, GT6839, or ET5262, respectively. Such in vitro assays may also be used as more general screens to identify chemicals that inhibit such enzymatic activity and that are therefore novel herbicide candidates. Alternatively, recombinantly produced proteins having ET1158, GT6839, or ET5262 activity may be used to elucidate the complex structure of these molecules and to further characterize their association with known inhibitors in order to rationally design new inhibitory herbicides as well as herbicide tolerant forms of the enzymes.

In Vitro Inhibitor Assays: Discovery of Small Molecule Ligand that Interacts with the Gene Product of SEQ ID NO:1, SEQ ID NO:3, or SEQ ID NO:5

Once a protein has been identified as a potential herbicide target, the next step is to develop an assay that allows screening large number of chemicals to determine which ones interact with the protein. Although it is straightforward to develop assays for proteins of known function, developing assays with proteins of unknown functions is more difficult.

This difficulty can be overcome by using technologies that can detect interactions between a protein and a compound without knowing the biological function of the protein. A short description of three methods is presented, including fluorescence correlation spectroscopy, surface-enhanced laser desorption/ionization, and biacore technologies.

Fluorescence Correlation Spectroscopy (FCS) theory was developed in 1972 but it is only in recent years that the technology to perform FCS became available (Madge et al. (1972) Phys. Rev. Lett., 29: 705-708; Maiti et al. (1997) Proc. Natl. Acad. Sci. USA, 94: 11753-11757). FCS measures the average diffusion rate of a fluorescent molecule within a small sample volume. The sample size can be as low as 10³ fluorescent molecules and the sample volume as low as the cytoplasm of a single bacterium. The diffusion rate is a function of the mass of the molecule and decreases as the mass increases. FCS can therefore be applied to protein-ligand interaction analysis by measuring the change in mass and therefore in diffusion rate of a molecule upon binding. In a typical experiment, the target to be analyzed is expressed as a recombinant protein with a sequence tag, such as a poly-histidine sequence, inserted at the N or C-terminus. The expression takes place in E. coli, yeast or insect cells. The protein is purified by chromatography. For example, the poly-histidine tag can be used to bind the expressed protein to a metal chelate column such as Ni2+ chelated on iminodiacetic acid agarose. The protein is then labeled with a fluorescent tag such as carboxytetramethylrhodamine or BODIPY® (Molecular Probes, Eugene, Oreg.). The protein is then exposed in solution to the potential ligand, and its diffusion rate is determined by FCS using instrumentation available from Carl Zeiss, Inc. (Thornwood, N.Y.). Ligand binding is determined by changes in the diffusion rate of the protein.

Surface-Enhanced Laser Desorption/Ionization (SELDI) was invented by Hutchens and Yip during the late 1980's (Hutchens and Yip (1993) Rapid Commun. Mass Spectrom. 7: 576-580). When coupled to a time-of-flight mass spectrometer (TOF), SELDI provides a mean to rapidly analyze molecules retained on a chip. It can be applied to ligand-protein interaction analysis by covalently binding the target protein on the chip and analyze by MS the small molecules that bind to this protein (Worrall et al. (1998) Anal. Biochem. 70: 750-756). In a typical experiment, the target to be analyzed is expressed as described for FCS. The purified protein is then used in the assay without further preparation. It is bound to the SELDI chip either by utilizing the poly-histidine tag or by other interaction such as ion exchange or hydrophobic interaction. The chip thus prepared is then exposed to the potential ligand via, for example, a delivery system capable to pipet the ligands in a sequential manner (autosampler). The chip is then submitted to washes of increasing stringency, for example a series of washes with buffer solutions containing an increasing ionic strength. After each wash, the bound material is analyzed by submitting the chip to SELDI-TOF. Ligands that specifically bind the target will be identified by the stringency of the wash needed to elute them.

Biacore relies on changes in the refractive index at the surface layer upon binding of a ligand to a protein immobilized on the layer. In this system, a collection of small ligands is injected sequentially in a 2-5 microlitre cell with the immobilized protein. Binding is detected by surface plasmon resonance (SPR) by recording laser light refracting from the surface. In general, the refractive index change for a given change of mass concentration at the surface layer, is practically the same for all proteins and peptides, allowing a single method to be applicable for any protein (Liedberg et al. (1983) Sensors Actuators 4: 299-304; Malmquist (1993) Nature, 361: 186-187). In a typical experiment, the target to be analyzed is expressed as described for FCS. The purified protein is then used in the assay without further preparation. It is bound to the Biacore chip either by utilizing the poly-histidine tag or by other interaction such as ion exchange or hydrophobic interaction. The chip thus prepared is then exposed to the potential ligand via the delivery system incorporated in the instruments sold by Biacore (Uppsala, Sweden) to pipet the ligands in a sequential manner (autosampler). The SPR signal on the chip is recorded and changes in the refractive index indicate an interaction between the immobilized target and the ligand. Analysis of the signal kinetics on rate and off rate allows the discrimination between non-specific and specific interaction.

Also, an assay for small molecule ligands that interact with a polypeptide is an inhibitor assay. For example, such an inhibitor assay useful for identifying inhibitors of the products essential plant genes, such as ET1158, GT6839, or ET5262 genes, comprises the steps of:

a) reacting an ET1158, GT6839, or ET5262 protein, respectively, and a substrate thereof in the presence of a suspected inhibitor of the protein's respective function;

b) comparing the rate of enzymatic activity of the protein in the presence of the suspected inhibitor to the rate of enzymatic activity under the same conditions in the absence of the suspected inhibitor; and

c) determining whether the suspected inhibitor inhibits the ET1158, GT6839, or ET5262 protein, respectively.

For example, the inhibitory effect on ET1158, GT6839, or ET5262 activity, may be determined by a reduction or complete inhibition of ET1158, GT6839, or ET5262 activity, respectively, in the assay. Such a determination may be made by comparing, in the presence and absence of the candidate inhibitor, the amount of substrate used or intermediate or product made during the reaction.

VI. In Vivo Inhibitor Assay

In one embodiment, a suspected herbicide, for example identified by in vitro screening, is applied to plants at various concentrations. The suspected herbicide is preferably sprayed on the plants. After application of the suspected herbicide, its effect on the plants, for example death or suppression of growth is recorded.

In another embodiment, an in vivo screening assay for inhibitors of the ET1158, GT6839, or ET5262 activity uses transgenic plants, plant tissue, plant seeds or plant cells capable of overexpressing a nucleotide sequence having ET1158, GT6839, or ET5262 activity, respectively, wherein the ET1158, GT6839, or ET5262 gene product is enzymatically active in the transgenic plants, plant tissue, plant seeds or plant cells. The nucleotide sequence is preferably derived from an eukaryote, such as a yeast, but is preferably derived from a plant. In a further preferred embodiment, the nucleotide sequence is identical or substantially similar to the nucleotide sequence set forth in SEQ ID NO:1, or encodes an enzyme having ET1158 activity, whose amino acid sequence is identical or substantially similar to the amino acid sequence set forth in SEQ ID NO:2. In another preferred embodiment, the nucleotide sequence is derived from a prokaryote. In a further embodiment, the nucleotide sequence is derived from an eukaryote, such as a yeast, but is preferably derived from a plant. In a preferred embodiment, the nucleotide sequence is identical or substantially similar to the nucleotide sequence set forth in SEQ ID NO:3, or encodes an enzyme having GT6839 activity, whose amino acid sequence is identical or substantially similar to the amino acid sequence set forth in SEQ ID NO:4. In another preferred embodiment, the nucleotide sequence is derived from a prokaryote. In a further preferred embodiment, the nucleotide sequence is derived from an eukaryote, such as a yeast, but is preferably derived from a plant. In a further preferred embodiment, the nucleotide sequence is identical or substantially similar to the nucleotide sequence set forth in SEQ ID NO:5, or encodes an enzyme having ET5262 activity, whose amino acid sequence is identical or substantially similar to the amino acid sequence set forth in SEQ ID NO:6. In another preferred embodiment, the nucleotide sequence is derived from a prokaryote.

A chemical is then applied to the transgenic plants, plant tissue, plant seeds or plant cells and to the isogenic non-transgenic plants, plant tissue, plant seeds or plant cells, and the growth or viability of the transgenic and non-transformed plants, plant tissue, plant seeds or plant cells are determined after application of the chemical and compared. Compounds capable of inhibiting the growth of the non-transgenic plants, but not affecting the growth of the transgenic plants are selected as specific inhibitors of ET1158, GT6839, or ET5262 activity.

VII. Herbicide Tolerant Plants

The present invention is further directed to plants, plant tissue, plant seeds, and plant cells tolerant to herbicides that inhibit the naturally occurring ET1158, GT6839, or ET5262 activity in these plants, wherein the tolerance is conferred by an altered ET1158, GT6839, or ET5262 activity, respectively. Altered ET1158, GT6839, or ET5262 activity may be conferred upon a plant according to the invention by increasing expression of wild-type herbicide-sensitive ET1158, GT6839, or ET5262 gene, respectively, for example by providing additional wild-type ET1158, GT6839, or ET5262 genes and/or by overexpressing the endogenous ET1158, GT6839, or ET5262 gene, for example by driving expression with a strong promoter. Altered ET1158, GT6839, or ET5262 activity also may be accomplished by expressing nucleotide sequences that are substantially similar to SEQ ID NO:1, SEQ ID NO:3, or SEQ ID NO:5, respectively, or homologs in a plant. Still further altered ET1158, GT6839, or ET5262 activity is conferred on a plant by expressing modified herbicide-tolerant ET1158, GT6839, or ET5262 genes, respectively, in the plant. Combinations of these techniques may also be used. Representative plants include any plants to which these herbicides are applied for their normally intended purpose. Preferred are agronomically important crops such as cotton, soybean, oilseed rape, sugar beet, maize, rice, wheat, barley, oats, rye, sorghum, millet, turf, forage, turf grasses, and the like.

A. Increased Expression of Wild-Type ET1158, GT6839, or ET5262

Achieving altered ET1158, GT6839, or ET5262 activity through increased expression results in a level of ET1158, GT6839, or ET5262 activity, respectively, in the plant cell at least sufficient to overcome growth inhibition caused by the herbicide when applied in amounts sufficient to inhibit normal growth of control plants. The level of expressed enzyme generally is at least two times, preferably at least five times, and more preferably at least ten times the natively expressed amount. Increased expression may be due to multiple copies of a wild-type ET1158, GT6839, or ET5262 gene; multiple occurrences of the coding sequence within the gene (i.e. gene amplification) or a mutation in the non-coding, regulatory sequence of the endogenous gene in the plant cell. Plants having such altered gene activity can be obtained by direct selection in plants by methods known in the art (see, e.g. U.S. Pat. No. 5,162,602, and U.S. Pat. No. 4,761,373, and references cited therein). These plants also may be obtained by genetic engineering techniques known in the art. Increased expression of a herbicide-sensitive ET1158, GT839, or ET5262 gene can also be accomplished by transforming a plant cell with a recombinant or chimeric DNA molecule comprising a promoter capable of driving expression of an associated structural gene in a plant cell operatively linked to a homologous or heterologous structural gene encoding the ET1158, GT6839, or ET5262 protein, respectively, or a homolog thereof. Preferably, the transformation is stable, thereby providing a heritable transgenic trait.

B. Expression of Modified Herbicide-Tolerant ET1158, GT6839, or ET5262 Proteins

According to this embodiment, plants, plant tissue, plant seeds, or plant cells are stably transformed with a recombinant DNA molecule comprising a suitable promoter functional in plants operatively linked to a coding sequence encoding a herbicide tolerant form of the ET1158, GT6839, or ET5262 protein. A herbicide tolerant form of the enzyme has at least one amino acid substitution, addition or deletion that confers tolerance to a herbicide that inhibits the unmodified, naturally occurring form of the enzyme. The transgenic plants, plant tissue, plant seeds, or plant cells thus created are then selected by conventional selection techniques, whereby herbicide tolerant lines are isolated, characterized, and developed. Below are described methods for obtaining genes that encode herbicide tolerant forms of ET1158, GT6839, or ET5262 protein:

One general strategy involves direct or indirect mutagenesis procedures on microbes. For instance, a genetically manipulatable microbe such as E. coli or S. cerevisiae may be subjected to random mutagenesis in vivo with mutagens such as UV light or ethyl or methyl methane sulfonate. Mutagenesis procedures are described, for example, in Miller, Experiments in Molecular Genetics, Cold Spring Harbor Laboratory, Cold Spring Harbor, N.Y. (1972); Davis et al., Advanced Bacterial Genetics, Cold Spring Harbor Laboratory, Cold Spring Harbor, N.Y. (1980); Sherman et al., Methods in Yeast Genetics, Cold Spring Harbor Laboratory, Cold Spring Harbor, N.Y. (1983); and U.S. Pat. No. 4,975,374. For example, the microbe selected for mutagenesis contains a normal, inhibitor-sensitive ET1158, GT6839, or ET5262 gene, or nucleotide sequence substantially similar thereto, which encodes a protein having ET1158, GT6839, or ET5262 gene product activity, and is dependent upon the activity conferred by this gene for growth. The mutagenized cells are grown in the presence of the inhibitor at concentrations that inhibit the unmodified gene. Colonies of the mutagenized microbe that grow better than the unmutagenized microbe in the presence of the inhibitor (i.e. exhibit resistance to the inhibitor) are selected for further analysis. ET1158, GT6839, or ET5262 genes conferring tolerance to the inhibitor are isolated from these colonies, either by cloning or by PCR amplification, and their sequences are elucidated. Sequences encoding altered gene products are then cloned back into the microbe to confirm their ability to confer inhibitor tolerance.

A method of obtaining mutant herbicide-tolerant alleles of a plant ET1158, GT6839, or ET5262 gene involves direct selection in plants. For example, the effect of a mutagenized ET1158, GT6839, or ET5262 gene on the growth inhibition of plants such as Arabidopsis, soybean, or maize is determined by plating seeds sterilized by art-recognized methods on plates on a simple minimal salts medium containing increasing concentrations of the inhibitor. Such concentrations are in the range of 0.001, 0.003, 0.01, 0.03, 0.1, 0.3, 1, 3, 10, 30, 110, 300, 1000 and 3000 parts per million (ppm). The lowest dose at which significant growth inhibition can be reproducibly detected is used for subsequent experiments. Determination of the lowest dose is routine in the art.

Mutagenesis of plant material is utilized to increase the frequency at which resistant alleles occur in the selected population. Mutagenized seed material is derived from a variety of sources, including chemical or physical mutagenesis or seeds, or chemical or physical mutagenesis or pollen (Neuffer, In Maize for Biological Research Sheridan, ed. Univ. Press, Grand Forks, N.Dak., pp. 61-64 (1982)), which is then used to fertilize plants and the resulting MI mutant seeds collected. Typically for Arabidopsis, M2 seeds (Lehle Seeds, Tucson, Ariz.), which are progeny seeds of plants grown from seeds mutagenized with chemicals, such as ethyl methane sulfonate, or with physical agents, such as gamma rays or fast neutrons, are plated at densities of up to 10,000 seeds/plate (10 cm diameter) on minimal salts medium containing an appropriate concentration of inhibitor to select for tolerance. Seedlings that continue to grow and remain green 7-21 days after plating are transplanted to soil and grown to maturity and seed set. Progeny of these seeds are tested for tolerance to the herbicide. If the tolerance trait is dominant, plants whose seed segregate 3:1/resistant:sensitive are presumed to have been heterozygous for the resistance at the M2 generation. Plants that give rise to all resistant seed are presumed to have been homozygous for the resistance at the M2 generation. Such mutagenesis on intact seeds and screening of their M2 progeny seed can also be carried out on other species, for instance soybean (see, e.g. U.S. Pat. No. 5,084,082). Alternatively, mutant seeds to be screened for herbicide tolerance are obtained as a result of fertilization with pollen mutagenized by chemical or physical means.

Confirmation that the genetic basis of the herbicide tolerance is a ET1158, GT6839, or ET5262 gene is ascertained as exemplified below. First, alleles of the ET1158, GT6839, or ET5262 gene from plants exhibiting resistance to the inhibitor are isolated using PCR with primers based either upon the Arabidopsis cDNA coding sequences shown in SEQ ID NO:1, SEQ ID NO:3, or SEQ ID NO:5, respectively, or, more preferably, based upon the unaltered ET1158, GT6839, or ET5262 gene sequence from the plant used to generate tolerant alleles. After sequencing the alleles to determine the presence of mutations in the coding sequence, the alleles are tested for their ability to confer tolerance to the inhibitor on plants into which the putative tolerance-conferring alleles have been transformed. These plants can be either Arabidopsis plants or any other plant whose growth is susceptible to the ET1158, GT6839, or ET5262 inhibitors. Second, the inserted ET1158, GT6839, or ET5262 genes are mapped relative to known restriction fragment length polymorphisms (RFLPs) (See, for example, Chang et al. Proc. Natl. Acad, Sci, USA 85: 6856-6860 (1988); Nam et al., Plant Cell 1: 699-705 (1989), cleaved amplified polymorphic sequences (CAPS) (Konieczny and Ausubel (1993) The Plant Journal, 4(2): 403-410), or SSLPs (Bell and Ecker (1994) Genomics, 19: 137-144). The ET1158, GT6839, or ET5262 inhibitor tolerance trait is independently mapped using the same markers. When tolerance is due to a mutation in that ET1158, GT6839, or ET5262 gene, the tolerance trait maps to a position indistinguishable from the position of the ET1158, GT6839, or ET5262 gene.

Another method of obtaining herbicide-tolerant alleles of a ET1158, GT6839, or ET5262 gene is by selection in plant cell cultures. Explants of plant tissue, e.g. embryos, leaf disks, etc. or actively growing callus or suspension cultures of a plant of interest are grown on medium in the presence of increasing concentrations of the inhibitory herbicide or an analogous inhibitor suitable for use in a laboratory environment. Varying degrees of growth are recorded in different cultures. In certain cultures, fast-growing variant colonies arise that continue to grow even in the presence of normally inhibitory concentrations of inhibitor. The frequency with which such faster-growing variants occur can be increased by treatment with a chemical or physical mutagen before exposing the tissues or cells to the inhibitor. Putative tolerance-conferring alleles of the ET1158, GT6839, or ET5262 gene are isolated and tested as described in the foregoing paragraphs. Those alleles identified as conferring herbicide tolerance may then be engineered for optimal expression and transformed into the plant. Alternatively, plants can be regenerated from the tissue or cell cultures containing these alleles.

Still another method involves mutagenesis of wild-type, herbicide sensitive plant ET1158, GT6839, or ET5262 genes in genetically manipulatable microbes, followed by culturing the microbe on medium that contains inhibitory concentrations (i.e. sufficient to cause abnormal growth, inhibit growth or cause cell death) of the inhibitor, and then selecting those colonies that grow normally in the presence of the inhibitor. More specifically, a plant cDNA, such as the Arabidopsis cDNA encoding the ET1158, GT6839, or ET5262 protein, is cloned into a microbe that is dependent on ET1158, GT6839, or ET5262 gene product activity, respectively, for growth, or that otherwise lacks the ET1158, GT6839, or ET5262 activity. The transformed microbe is then subjected to in vivo mutagenesis or to in vitro mutagenesis by any of several chemical or enzymatic methods known in the art, e.g. sodium bisulfite (Shortle et al., Methods Enzymol. 100:457-468 (1983); methoxylamine (Kadonaga et al., Nucleic Acids Res. 13:1733-1745 (1985); oligonucleotide-directed saturation mutagenesis (Hutchinson et al., Proc. Natl. Acad. Sci. USA, 83:710-714 (1986); or various polymerase misincorporation strategies (see, e.g. Shortle et al., Proc. Natl. Acad. Sci. USA, 79:1588-1592 (1982); Shiraishi et al., Gene 64:313-319 (1988); and Leung et al., Technique 1:11-15 (1989). Colonies that grow normally in the presence of normally inhibitory concentrations of inhibitor are picked and purified by repeated restreaking. Their plasmids are purified and tested for the ability to confer tolerance to the inhibitor by retransforming them into the microbe lacking ET1158, GT6839, or ET5262 activity, respectively. The DNA sequences of cDNA inserts from plasmids that pass this test are then determined.

Herbicide resistant ET1158, GT6839, or ET5262 proteins are also obtained using methods involving in vitro recombination, also called DNA shuffling. By DNA shuffling, mutations, preferably random mutations, are introduced into nucleotide sequences encoding ET1158, GT6839, or ET5262 activity. DNA shuffling also leads to the recombination and rearrangement of sequences within a ET1158, GT6839, or ET5262 gene or to recombination and exchange of sequences between two or more different of ET1158, GT6839, or ET5262 genes. These methods allow for the production of millions of mutated ET1158, GT6839, or ET5262 coding sequences. The mutated genes, or shuffled genes, are screened for desirable properties, e.g. improved tolerance to herbicides and for mutations that provide broad spectrum tolerance to the different classes of inhibitor chemistry. Such screens are well within the skills of a routineer in the art.

In a preferred embodiment, a mutagenized ET1158, GT6839, or ET5262 gene is formed from at least one template ET1158, GT6839, or ET5262 gene, wherein the template ET1158, GT6839, or ET5262 gene has been cleaved into double-stranded random fragments of a desired size, and comprising the steps of adding to the resultant population of double-stranded random fragments one or more single or double-stranded oligonucleotides, wherein said oligonucleotides comprise an area of identity and an area of heterology to the double-stranded random fragments; denaturing the resultant mixture of double-stranded random fragments and oligonucleotides into single-stranded fragments; incubating the resultant population of single-stranded fragments with a polymerase under conditions which result in the annealing of said single-stranded fragments at said areas of identity to form pairs of annealed fragments, said areas of identity being sufficient for one member of a pair to prime replication of the other, thereby forming a mutagenized double-stranded polynucleotide; and repeating the second and third steps for at least two further cycles, wherein the resultant mixture in the second step of a further cycle includes the mutagenized double-stranded polynucleotide from the third step of the previous cycle, and the further cycle forms a further mutagenized double-stranded polynucleotide, wherein the mutagenized polynucleotide is a mutated ET1158, GT6839, or ET5262 gene having enhanced tolerance to a herbicide which inhibits naturally occurring ET1158, GT6839, or ET5262 activity, respectively. In a preferred embodiment, the concentration of a single species of double-stranded random fragment in the population of double-stranded random fragments is less than 1% by weight of the total DNA. In a further preferred embodiment, the template double-stranded polynucleotide comprises at least about 100 species of polynucleotides. In another preferred embodiment, the size of the double-stranded random fragments is from about 5 bp to 5 kb. In a further preferred embodiment, the fourth step of the method comprises repeating the second and the third steps for at least 10 cycles. Such method is described e.g. in Stemmer et al. (1994) Nature 370: 389-391, in U.S. Pat. No. 5,605,793, U.S. Pat. No. 5,811,238 and in Crameri et al. (1998) Nature 391: 288-291, as well as in WO 97/20078, and these references are incorporated herein by reference.

In another preferred embodiment, any combination of two or more different ET1158, GT6839, or ET5262 genes are mutagenized in vitro by a staggered extension process (StEP), as described e.g. in Zhao et al. (1998) Nature Biotechnology 16: 258-261. The two or more ET1158, GT6839, or ET5262 genes are used as template for PCR amplification with the extension cycles of the PCR reaction preferably carried out at a lower temperature than the optimal polymerization temperature of the polymerase. For example, when a thermostable polymerase with an optimal temperature of approximately 72° C. is used, the temperature for the extension reaction is desirably below 72° C., more desirably below 65° C., preferably below 60° C., more preferably the temperature for the extension reaction is 55° C. Additionally, the duration of the extension reaction of the PCR cycles is desirably shorter than usually carried out in the art, more desirably it is less than 30 seconds, preferably it is less than 15 seconds, more preferably the duration of the extension reaction is 5 seconds. Only a short DNA fragment is polymerized in each extension reaction, allowing template switch of the extension products between the starting DNA molecules after each cycle of denaturation and annealing, thereby generating diversity among the extension products. The optimal number of cycles in the PCR reaction depends on the length of the ET1158, GT6839, or ET5262 genes to be mutagenized but desirably over 40 cycles, more desirably over 60 cycles, preferably over 80 cycles are used. Optimal extension conditions and the optimal number of PCR cycles for every combination of ET1158, GT6839, or ET5262 genes are determined as described in using procedures well-known in the art. The other parameters for the PCR reaction are essentially the same as commonly used in the art. The primers for the amplification reaction are preferably designed to anneal to DNA sequences located outside of the ET1158, GT6839, or ET5262 genes, e.g. to DNA sequences of a vector comprising the ET1158, GT6839, or ET5262 genes, whereby the different ET1158, GT6839, or ET5262 genes used in the PCR reaction are preferably comprised in separate vectors. The primers desirably anneal to sequences located less than 500 bp away from ET1158, GT6839, or ET5262 sequences, preferably less than 200 bp away from the ET1158, GT6839, or ET5262 sequences, more preferably less than 120 bp away from the ET1158, GT6839, or ET5262 sequences. Preferably, the ET1158, GT6839, or ET5262 sequences are surrounded by restriction sites, which are included in the DNA sequence amplified during the PCR reaction, thereby facilitating the cloning of the amplified products into a suitable vector. In another preferred embodiment, fragments of ET1158, GT6839, or ET5262 genes having cohesive ends are produced as described in WO 98/05765. The cohesive ends are produced by ligating a first oligonucleotide corresponding to a part of a ET1158, GT6839, or ET5262 gene to a second oligonucleotide not present in the gene or corresponding to a part of the gene not adjoining to the part of the gene corresponding to the first oligonucleotide, wherein the second oligonucleotide contains at least one ribonucleotide. A double-stranded DNA is produced using the first oligonucleotide as template and the second oligonucleotide as primer. The ribonucleotide is cleaved and removed. The nucleotide(s) located 5′ to the ribonucleotide is also removed, resulting in double-stranded fragments having cohesive ends. Such fragments are randomly reassembled by ligation to obtain novel combinations of gene sequences.

Any ET1158, GT6839, or ET5262 gene or any combination of ET1158, GT6839, or ET5262 genes, or homologs thereof, is used for in vitro recombination in the context of the present invention, for example, a ET1158, GT6839, or ET5262 gene derived from a plant, such as, e.g. Arabidopsis thaliana, e.g. a ET1158 gene set forth in SEQ ID NO:1, a GT6839 gene set forth in SEQ ID NO:3, and a ET5262 gene set forth in SEQ ID NO:5. Whole ET1158, GT6839, or ET5262 genes or portions thereof are used in the context of the present invention. The library of mutated ET1158, GT6839, or ET5262 genes obtained by the methods described above are cloned into appropriate expression vectors and the resulting vectors are transformed into an appropriate host, for example a plant cell, an algae like Chlamydomonas, a yeast or a bacteria. An appropriate host requires ET1158, GT6839, or ET5262 gene product activity for growth. Host cells transformed with the vectors comprising the library of mutated ET1158, GT6839, or ET5262 genes are cultured on medium that contains inhibitory concentrations of the inhibitor and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined.

An assay for identifying a modified ET1158, GT6839, or ET5262 gene that is tolerant to an inhibitor may be performed in the same manner as the assay to identify inhibitors of the ET1158, GT6839, or ET5262 activity (Inhibitor Assay, above) with the following modifications: First, a mutant ET1158, GT6839, or ET5262 protein is substituted in one of the reaction mixtures for the wild-type ET1158, GT6839 or ET5262 protein of the inhibitor assay. Second, an inhibitor of wild-type enzyme is present in both reaction mixtures. Third, mutated activity (activity in the presence of inhibitor and mutated enzyme) and unmutated activity (activity in the presence of inhibitor and wild-type enzyme) are compared to determine whether a significant increase in enzymatic activity is observed in the mutated activity when compared to the unmutated activity. Mutated activity is any measure of activity of the mutated enzyme while in the presence of a suitable substrate and the inhibitor. Unmutated activity is any measure of activity of the wild-type enzyme while in the presence of a suitable substrate and the inhibitor.

In addition to being used to create herbicide-tolerant plants, genes encoding herbicide-tolerant ET1158, GT6839, or ET5262 protein can also be used as selectable markers in plant cell transformation methods. For example, plants, plant tissue, plant seeds, or plant cells transformed with a heterologous DNA sequence can also be transformed with a sequence encoding an altered ET1158, GT6839, or ET5262 activity capable of being expressed by the plant. The transformed cells are transferred to medium containing an inhibitor of the enzyme in an amount sufficient to inhibit the growth or survivability of plant cells not expressing the modified coding sequence, wherein only the transformed cells will grow. The method is applicable to any plant cell capable of being transformed with a modified ET 1158, GT6839, or ET5262 gene, and can be used with any heterologous DNA sequence of interest. Expression of the heterologous DNA sequence and the modified gene can be driven by the same promoter functional in plant cells, or by separate promoters.

VIII. Plant Transformation Technology

A wild type or herbicide-tolerant form of the ET1158, GT6839, or ET5262 gene, or homologs thereof, can be incorporated in plant or bacterial cells using conventional recombinant DNA technology. Generally, this involves inserting a DNA molecule encoding the ET1158, GT6839, or ET5262 gene into an expression system to which the DNA molecule is heterologous (i.e., not normally present) using standard cloning procedures known in the art. The vector contains the necessary elements for the transcription and translation of the inserted protein-coding sequences in a host cell containing the vector. A large number of vector systems known in the art can be used, such as plasmids, bacteriophage viruses and other modified viruses. The components of the expression system may also be modified to increase expression. For example, truncated sequences, nucleotide substitutions, nucleotide optimization or other modifications may be employed. Expression systems known in the art can be used to transform virtually any crop plant cell under suitable conditions. A heterologous DNA sequence comprising a wild-type or herbicide-tolerant form of the ET1158, GT6839, or ET5262 gene is preferably stably transformed and integrated into the genome of the host cells. In another preferred embodiment, the heterologous DNA sequence comprising a wild-type or herbicide-tolerant form of the ET1158, GT6839, or ET5262 gene located on a self-replicating vector. Examples of self-replicating vectors are viruses, in particular gemini viruses. Transformed cells can be regenerated into whole plants such that the chosen form of the ET1158, GT6839, or ET5262 gene confers herbicide tolerance in the transgenic plants.

A. Requirements for Construction of Plant Expression Cassettes

Gene sequences intended for expression in transgenic plants are first assembled in expression cassettes behind a suitable promoter expressible in plants. The expression cassettes may also comprise any further sequences required or selected for the expression of the heterologous DNA sequence. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described infra. The following is a description of various components of typical expression cassettes.

1. Promoters

The selection of the promoter used in expression cassettes will determine the spatial and temporal expression pattern of the heterologous DNA sequence in the plant transformed with this DNA sequence. Selected promoters will express heterologous DNA sequences in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters known in the art can be used. For example, for constitutive expression, the CaMV 35S promoter, the rice actin promoter, or the ubiquitin promoter may be used. For regulatable expression, the chemically inducible PR-1 promoter from tobacco or Arabidopsis may be used (see, e.g., U.S. Pat. No. 5,689,044).

2. Transcriptional Terminators

A variety of transcriptional terminators are available for use in expression cassettes. These are responsible for the termination of transcription beyond the heterologous DNA sequence and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledonous and dicotyledonous plants.

3. Sequences for the Enhancement or Regulation of Expression

Numerous sequences have been found to enhance gene expression from within the transcriptional unit and these sequences can be used in conjunction with the genes of this invention to increase their expression in transgenic plants. For example, various intron sequences such as introns of the maize AdhI gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene optionally is genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al., Bio/technol. 11: 194 (1993); Fennoy and Bailey-Serres. Nucl. Acids Res. 21: 5294-5300 (1993). Methods for modifying coding sequences by taking into account codon usage in plant genes and in higher plants, green algae, and cyanobacteria are well known (see table 4 in: Murray et al. Nucl. Acids Res. 17: 477-498 (1989); Campbell and Gowri Plant Physiol. 92: 1-11(1990).

5. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous products encoded by DNA sequences to these organelles. In addition, sequences have been characterized which cause the targeting of products encoded by DNA sequences to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)). By the fusion of the appropriate targeting sequences described above to heterologous DNA sequences of interest it is possible to direct this product to any organelle or cell compartment.

B. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptII gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), the manA gene, which allows for positive selection in the presence of mannose (Miles and Guest (1984) Gene, 32:41-48; U.S. Pat. No. 5,767,378), the dhfr gene, which confers resistance to methotrexate (Bourouis et al., EMBO J. 2(7): 1099-1104 (1983)), and the EPSPS gene, which confers resistance to glyphosate (U.S. Pat. Nos. 4,940,935 and 5,188,642).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Typical vectors suitable for Agrobacterium transformation include the binary vectors pCIB200 and pCIB2001, as well as the binary vector pCIB 10 and hygromycin selection derivatives thereof. (See, for example, U.S. Pat. No. 5,639,949).

2. Vectors Suitable for non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Typical vectors suitable for non-Agrobacterium transformation include pCIB3064, pSOG19, and pSOG35. (See, for example, U.S. Pat. No. 5,639,949).

C. Transformation Techniques

Once the coding sequence of interest has been cloned into an expression system, it is transformed into a plant cell. Methods for transformation and regeneration of plants are well known in the art. For example, Ti plasmid vectors have been utilized for the delivery of foreign DNA, as well as direct DNA uptake, liposomes, electroporation, micro-injection, and microprojectiles. In addition, bacteria from the genus Agrobacterium can be utilized to transform plant cells.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. In each case the transformed cells are regenerated to whole plants using standard techniques known in the art.

Transformation of most monocotyledon species has now also become routine. Preferred techniques include direct gene transfer into protoplasts using PEG or electroporation techniques, particle bombardment into callus tissue, as well as Agrobacterium-mediated transformation.

D. Plastid Transformation

In another preferred embodiment, a nucleotide sequence encoding a polypeptide having ET1158, GT6839, or ET5262 activity is directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit expression levels that can readily exceed 10% of the total soluble plant protein. In a preferred embodiment, the nucleotide sequence is inserted into a plastid targeting vector and transformed into the plastid genome of a desired plant host. Plants homoplasmic for plastid genomes containing the nucleotide sequence are obtained, and are preferentially capable of high expression of the nucleotide sequence.

Plastid transformation technology is for example extensively described in U.S. Pat. Nos. 5,451,513, 5,545,817, 5,545,818, and 5,877,462 in PCT application no. WO 95/16783 and WO 97/32977, and in McBride et al. (1994) Proc. Natl. Acad. Sci. USA 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab, Z., Hajdukiewicz, P., and Maliga, P. (1990) Proc. Natl. Acad. Sci. USA 87, 8526-8530; Staub, J. M., and Maliga, P. (1992) Plant Cell 4, 39-45). The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab, Z., and Maliga, P. (1993) Proc. Natl. Acad. Sci. USA 90, 913-917). Other selectable markers useful for plastid transformation are known in the art and encompassed within the scope of the invention.

IX. Breeding

The wild-type or altered form of a ET1158, GT6839, or ET5262 gene of the present invention can be utilized to confer herbicide tolerance to a wide variety of plant cells, including those of gymnosperms, monocots, and dicots. Although the gene can be inserted into any plant cell falling within these broad classes, it is particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

The high-level expression of a wild-type ET1158, GT6839, or ET5262 gene and/or the expression of herbicide-tolerant forms of a ET1158, GT6839, or ET5262 gene conferring herbicide tolerance in plants, in combination with other characteristics important for production and quality, can be incorporated into plant lines through breeding approaches and techniques known in the art.

Where a herbicide tolerant ET1158, GT6839, or ET5262 gene allele is obtained by direct selection in a crop plant or plant cell culture from which a crop plant can be regenerated, it is moved into commercial varieties using traditional breeding techniques to develop a herbicide tolerant crop without the need for genetically engineering the allele and transforming it into the plant.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by Sambrook, et al., Molecular Cloning, eds., Cold Spring Harbor Laboratory Press, Cold Spring Harbor, N.Y. (1989) and by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987), Reiter, et al., Methods in Arabidopsis Research, World Scientific Press (1992), and Schultz et al., Plant Molecular Biology Manual, Kluwer Academic Publishers (1998). These references describe the standard techniques used for all steps in tagging and cloning genes from Ac/Ds transposon mutagenized populations of Arabidopsis: plant infection and transformation; screening for the identification of seedling mutants; and cosegregation analysis. Ds transposon insertion lines produced as described in Sundareson et al. (1995) Genes and Dev., 9:1797-1810) were used in these experiments.

### Example 1

### Transposon Border Isolation

Arabidopsis genomic DNA is isolated from line ET1158, GT6839, or ET5262 using the Nucleon PhytoPure™ Plant DNA Isolation Kit (Amersham International plc, Buckinghamshire, England). Fragments of genomic DNA flanking the borders of the transposon are isolated using the TAIL-PCR technique (Liu et al. (1995) The Plant Journal, 8:457-463; Liu and Whittier (1995), Genomics, 25: 674-681). Three sets of 12 TAIL-PCR reactions, referred to as the primary, secondary and tertiary reactions, are performed. In each reaction, one arbitrary degenerate primer and one transposon-specific primer are used. The arbitrary degenerate primer is chosen from among six primers, LWAD1, CA51, CA52, CA53, CA54, and CA55 (Table 1), which are used to prime the genomic DNA flanking the insertion. These degenerate primers are used in combination with two sets of three, nested, transposon-specific primers (Table 2). These primers are homologous to regions of the Ds elements which lie at the outermost ends of the transposons, DS5 at the 5′ end (primers 5A, 5B, and 5C) and DS3 at the 3′ end primers 3A, 3B, and 3C). When the degenerate and nested primer pairs are used in a series of low and high-stringency PCR amplifications, as described in the TAIL-PCR protocol (Liu and Whittier (1995), Genomics, 25: 674-681), DNA fragments are produced which correspond to the genomic DNA that is directly adjacent to the transposon insertion. The nucleic acid sequence of the PCR products from the tertiary TAIL-PCR reactions are then determined by standard molecular biology techniques. The resulting sequences are analyzed for the presence of non-Ds transposon vector sequence. To confirm the integrity of the resultant products, PCR primers specific to the flanking genomic region are designed and used in conjunction with the tertiary nested primer in a PCR reaction, to confirm the transposon insertion point within the genomic DNA. Finding a PCR product of the appropriate size, based on the sequence of the TAIL-PCR clone confirms a valid rescue.

TABLE 1
DEGENERATE PRIMERS

| ID NO | PRIMER | DEGEN. | PRIMER SEQUENCE | NOTES AND REFERENCES |
|---|---|---|---|---|
| 7 | LWAD1 | 1026 | NGT TGW GNA TWT SGW GNT | designed by L. Wegrich |
| 8 | CA51 | 128 | TGW GNA GSA NCA SAG | derivative of primer AD1₍₂₎ |
| 9 | CA52 | 128 | AGW GNA GWA NCA WAG G | identical to primer AD2₍₂₎ |
| 10 | CA53 | 256 | STT GNT AST NCT NTG C | identical to primer AD5₍₃₎ |
| 11 | CA54 | 64 | NTC GAS TWT SGW GTT | identical to primer AD1₍₁₎ |
| 12 | CA55 | 256 | WGT GNA GWA NCA NAG A | identical to primer AD3₍₁₎ |

TABLE 2
NESTED PRIMERS

| ID NO | PRIMER | PRIMER SEQUENCE | NOTES |
|---|---|---|---|
| 13 | 5A | ACTAGCTCTACCGTTTCCGTTTCCGTTTAC | DS5 PRIMARY |
| 14 | 5B | TTACCTCGGGTTCGAAATCGATCGGGATAA | DS5 SECONDARY |
| 15 | 5C | AAAATCGGTTATACGATAACGGTCGGTACGGGA | DS5 TERTIARY |
| 16 | 3A | GGGTCTTGCGGATCTGAATATATGTTTTCATGTG | DS3 PRIMARY |
| 17 | 3B | TACCGAAGAAAAATACCGGTTCCCGTCCGATTTCGAC | DS3 SECONDARY |
| 18 | 3C | GGATCGTATCGGTTTTCGATTACCGTATTTATCC | DS3 TERTIARY |

### REFERENCES

1. Liu et al. (1995) The Plant Journal, 8:457-463

2. Liu and Whittier (1995) Genomics, 25: 674-681

3. Tsugeki et al. (1996) The Plant Journal, 10: 479-489

### Example 2

### Sequence Analysis of Tagged Seedling Lethal Line ET1158

For transposant line ET1158, four PCR products are obtained from the DS3 border and one product from the DS5 border. The preliminary sequences obtained from the TAIL-PCR border products from transposant line ET1158 are used in BLASTn searches against nucleotide databases (Altschul et al. (1990) J Mol. Biol. 215:403-410; Altschul et al. (1997) Nucleic Acids Res. 25:3389-3402). The initial sequence obtained for the region bordering the Ds3 end of the transposon has a 98.7% identity to bases 78174 through 78698 of Arabidopsis chromosome 2, BAC F504 (Genbank accession number AC005936). The initial sequence of the region bordering the Ds5 end has 97.8% identity to bases 77674 through 78173 of BAC F504, and include a nine base insertion (GATGATGAT) between bases 77982/83, differing from the published sequence. Further analysis of the border sequences reveals a nine base pair duplication that occurred during the transposon insertion, corresponding to bases 78,165 through 78,173 of BAC F504. This region of BAC F504 is annotated as encoding a putative DNA-binding protein (Genbank accession number ACC97225).

To identify the ORF for this gene, primers are designed to the 5′ and 3′ ends of the predicted ORF for a putative DNA-binding protein. PCR is performed using template DNA from the pFL61 Arabidopsis cDNA library (Minet et al. (1992) Plant J. 2: 417-422). The resulting PCR product is TA-cloned (Original TA-Cloning kit, Invitrogen) and sequenced. The cDNA sequence is the same as the sequence predicted in the Genbank annotation, thus validating for the first time the putative open reading frame annotation.

A few polymorphisms are detected between A. thaliana ecotype Landsberg erecta and Columbia and are shown in the table below.

| cDNA base | ET1158 cDNA base | Columbia genomic base |
|---|---|---|
| 709 | C | A |
| 728 | A | G |
| 764 | C | G |
| 788 | T | C |
| 924-934 | CAT (3× repeat) | absent |

Further inspection of the ET1158 insertion event reveals that the transposon inserted near the 3′ end of the coding region for a putative DNA binding protein, annotated as gene F504.16.

Analysis of the DNA sequence from this gene reveals a high degree of sequence similarity to other putative zinc finger proteins from Arabidopsis thaliana (Genbank accession numbers AAD20087, AAC78253, AAD10684, AAC97277), as well as other plant species, including potato (Solanum tuberosum, Genbank accession number S48856) and maize (Zea mays, Genbank accession number ACC18941).

ET1158 HOMOLOGS

| DESCRIPTION | ACCESSION | DATABASE | % ID |
|---|---|---|---|
| Cpo 61.1-fruit fly | CAA78696.1 | GENBANK | 20.0 |
| (Drosophila melanogaster)* | | | |
| Zn f.p. ZFP108-house mouse | AAD45924 | GENBANK | 24.3 |
| (Mus musculus)* | | | |
| Zn f.p. 94-Chinese Hamster | AAC53577 | GENBANK | 24.6 |
| (Cricetulus griseus) | | | |
| HZF6, Krueppel-related finger | S47068 | PIR | 24.9 |
| protein (fragment) - Human | | | |
| (Homo sapiens) | | | |
| Putative DNA-binding protein | AAD20087 | GENBANK | 34.0 |
| (Arabidopsis thaliana)* | | | |
| Finger protein pcp1-potato | S48856 | PIR | 41.3 |
| (Solanum tuberosum) | | | |
| Putative Zn finger protein | AAC78253 | GENBANK | 44.5 |
| (Arabidopsis thaliana)* | | | |
| Zn finger protein-maize | ACC18941 | GENBANK | 45.9 |
| (Zea mays)* | | | |
| Putative DNA-binding protein | AAD10684 | GENBANK | 48.5 |
| (Arabidopsis thaliana)* | | | |
| Putative DNA-binding protein | AAC97277 | GENBANK | 56.3 |
| (Arabidopsis thaliana)* | | | |

*CONCEPTUAL TRANSLATION: amino acid sequence listed has been predicted based on a translation of the nucleotide sequence.
% ID: percent identity relative to the protein encoded by the Arabidopsis thaliana ET1158 gene

### Example 3

### Sequence Analysis of Tagged Seedling Lethal Line GT6839

For transposant line GT6839, one PCR product is obtained from the Ds3 border and one product from the Ds5 border. The preliminary sequences obtained from the TAIL-PCR border products from transposant line GT6839 are used in BLASTn searches against nucleotide databases (Altschul et al. (1990) J Mol. Biol. 215:403-410; Altschul et al. (1997) Nucleic Acids Res. 25:3389-3402). The initial sequence of the region bordering the Ds5 end indicates that the transposon has inserted with the Ds5 end adjacent to Arabidopsis genomic DNA (base numbers 84111 and higher of Arabidopsis chromosome 2, section 1, GenBank accession number AC006837). The initial sequence of the region bordering the Ds3 end indicates that the transposon has inserted with the Ds3 end adjacent to Arabidopsis genomic DNA (base numbers 84110 and lower of Arabidopsis chromosome 2, section 1). Analysis of the sequence from line GT6839 indicates that the Ds3 end of the transposon inserted 24 bp upstream of the initiator codon for an ORF encoding the Arabidopsis thaliana TatC gene (Genbank accession number AF145045).

Two other seedling lethal lines are identified with transposon insertions at different locations in this region, transposant lines GT8096 and ET7536.

For transposant line GT8096, two PCR products are obtained from the Ds5 border. The preliminary sequences obtained from the TAIL-PCR border products from transposant line GT8096 are used in BLASTn searches against nucleotide databases (Altschul et al. (1990) J Mol. Biol. 215:403-410; Altschul et al. (1997) Nucleic Acids Res. 25:3389-3402). The initial sequences of the region bordering the Ds5 end indicate that the transposon has inserted with the Ds5 end adjacent to Arabidopsis genomic DNA (base numbers 83660 and lower of Arabidopsis chromosome 2, section 1, GenBank accession number AC006837).

For transposant line ET7536, one PCR product is obtained from the Ds3 border and two products from the Ds5 border. The preliminary sequences obtained from the TAIL-PCR border products from transposant line ET7536 are used in BLASTn searches against nucleotide databases (Altschul et al. (1990) J Mol. Biol. 215:403-410; Altschul et al. (1997) Nucleic Acids Res. 25:3389-3402). The initial sequences of the region bordering the Ds5 end indicate that the transposon has inserted with the Ds5 end adjacent to Arabidopsis genomic DNA (base numbers 84173 and lower of Arabidopsis chromosome 2, section 1, GenBank accession number AC006837). The initial sequence of the region bordering the Ds3 end indicates that the transposon has inserted with the Ds3 end adjacent to Arabidopsis genomic DNA (base numbers 84166 and higher of Arabidopsis chromosome 2, section 1).

Analysis of the DNA sequence from the Arabidopsis thaliana TatC gene reveals a high degree of sequence similarity to other proteins which may play a role in chloroplast protein import, including Ycf43 (Synechococcus, Genbank accession number AAD26593), hypothetical protein 27.8 kd (Synechocystis, SWISS PROT accession number P54086), hypothetical protein 28.1 kd (Porhpyra purpurea, SWISS PROT accession number P51264), hypothetical chloroplast RF43 (Guillardia theta, Genbank accession number AAC35684), hypothetical protein 30.1 kd YCF43 (Odontella sinensis, SWISS PROT accession number P49538), E. coli TATC (SWISS PROT accession number P27857), and hypothetical protein (Haemophilus influenzae, SWISS PROT accession number P44560).

Primers are designed to the 5′ and 3′ ends of the published Arabidopsis thaliana Tat C cDNA. PCR is performed using template DNA from the pFL61 Arabidopsis cDNA library (Minet et al. (1992) Plant J. 2: 417-422). The resulting PCR product is TA-cloned (Original TA-Cloning kit, Invitrogen) and sequenced. The sequence data shows the cDNA to be the TatC cDNA.

| DESCRIPTION | ACCESSION | DATABASE | % ID |
|---|---|---|---|
| Ycf43 | AAD26593 | GENBANK | 58.7 |
| (Synechococcus)* | | | |
| Hypothetical Protein 27.8 KD | P54086 | SWISS PROT | 58.6 |
| (Synechocystis)* | | | |
| Hypothetical Protein 28.1 KD | P51264 | SWISS PROT | 45.2 |
| (Porhpyra purpurea)* | | | |
| Hypothetical Chloroplast | AAC35684 | GENBANK | 43.9 |
| RF 43 (Guillardia theta)* | | | |
| Hypothetical Protein 30.1 KD | P49538 | SWISS PROT | 43.4 |
| YCF 43 (Odontella sinensis)* | | | |
| E. coli TATC | P27857 | SWISS PROT | 35.7 |
| Hypothetical Protein | P44560 | SWISS PROT | 30.2 |
| (Haemophilus influenzae)* | | | |

*CONCEPTUAL TRANSLATION amino acid sequence listed has been predicted based on a translation of the nucleotide sequence.
% ID: percent identity relative to the protein encoded by the Arabidopsis thaliana GT6839 gene

### Example 4

### Sequence Analysis of Tagged Seedling Lethal Line ET5262

For transposant line ET5262, four PCR products are obtained from the DS3 border and four products from the DS5 border. These genomic sequences from Ds5 and Ds3 TAIL PCR products, excluding the nine base pair duplication created by the Ds insertion, are assembled together and listed in SEQ ID NO:21. The preliminary sequences obtained from the TAIL-PCR border products from transposant line ET5262 are used in BLASTn searches against nucleotide databases (Altschul et al. (1990) J Mol. Biol. 215:403-410; Altschul et al. (1997) Nucleic Acids Res. 25:3389-3402). Arabidopsis genomic DNA sequences for this gene are not identified in these searches.

The TAIL derived genomic sequence, corresponding to interrupted ET5262 ORF, is used to design primers for 5′ and 3′ RACE. 5′ and 3′ RACE is performed using the Marathon™ cDNA amplification kit (Clontech). The 5′ and 3′ race products are TA-cloned (Original TA-Cloning kit, Invitrogen) and sequenced. Sequence data are used to design 5′ and 3′ ET5262 cDNA primers. PCR is performed using these primers and template DNA from the pFL61 Arabidopsis cDNA library (Minet et al. (1992) Plant J. 2: 417-422). The resulting PCR product is TA-cloned and sequenced. Sequence data indicates that the cloned cDNA (SEQ ID NO:1) corresponds to the ORF interrupted in ET5262.

Analysis of the transposon insertion indicates that the Ds3 end of the transposon inserted at base 283/284 of the cDNA sequence, whereas the Ds5 end of the transposon inserted at base 274/275. The insertion resulted in a nine base pair duplication (CTTCTCAGC) corresponding to bases 275 through 283 of the cDNA.

Analysis of the DNA sequence from this gene reveals a high degree of sequence similarity to other putative proteins similar to 1-3 beta endoglucanases from Arabidopsis thaliana (Genbank accession numbers CAB41118 and AAB97119), as well as other plant species, including wheat (Triticum aestivum, SWISS PROT accession number P52409) and rape (Brassica napus, PIR accession number S31612).

ET5262 HOMOLOGS

| DESCRIPTION | ACCESSION | DATABASE | % ID |
|---|---|---|---|
| pHR2 - yeast | O13318 | SWISS PROT | 19.6 |
| (Candida albicans) | | | |
| Similar to S. cerevisiae | BAA13864 | GENBANK | 23.6 |
| glycolipid anchored surface | | | |
| protein precursor (S. pombe)* | | | |
| EPD1 precursor-yeast | P56092 | GENBANK | 24.3 |
| (Candida maltosa) | | | |
| Notch homolog-green blowfly | ACC36152 | GENBANK | 24.8 |
| (Lucilia cuprina) | | | |
| beta-1-3 glucanase (A20) - Rape | S31612 | PIR | 27.5 |
| (Brassica napus) | | | |
| (FRAGMENT)* | | | |
| Putative beta -1,3 endoglucanase | AAB97119 | GENBANK | 32.7 |
| (Arabidopsis thaliana)* | | | |
| Glucan endo 1-3 beta- | P52409 | SWISS PROT | 37.1 |
| glucosidase precursor-wheat | | | |
| (Triticum aestivum) | | | |
| Putative beta -1,3 endoglucanase | AAD17427 | GENBANK | 41.2 |
| (Arabidopsis thaliana)* | | | |
| Putative protein similar to | CAB41118 | GENBANK | 42.5 |
| beta -1,3 endoglucanase from | | | |
| wheat (Arabidopsis thaliana)* | | | |

* CONCEPTUAL TRANSLATION amino acid sequence listed has been predicted based on a translation of the nucleotide sequence.
% ID: percent identity relative to the protein encoded by the Arabidopsis thaliana ET5262 gene

### Example 5

### Expression of Recombinant ET1158, GT6839, or ET5262 Protein in E. coli

The coding region of the protein, corresponding to the cDNA clone SEQ ID NO:1 is subcloned into an appropriate expression vector, and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of the ET1158 activity is confirmed. Protein conferring ET1158 activity is isolated using standard techniques.

The coding region of the protein, corresponding to the cDNA clone SEQ ID NO:3 is subcloned into an appropriate expression vector, and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of the GT6839 activity is confirmed. Protein conferring GT6839 activity is isolated using standard techniques.

The coding region of the protein, corresponding to the eDNA clone SEQ ID NO:5, is subcloned into an appropriate expression vector, and transformed into E. coli using the manufacturer's conditions. Specific examples include plasmids such as pBluescript (Stratagene, La Jolla, Calif.), pFLAG (International Biotechnologies, Inc., New Haven, Conn.), and pTrcHis (Invitrogen, La Jolla, Calif.). E. coli is cultured, and expression of the ET5262 activity is confirmed. Protein conferring ET5262 activity is isolated using standard techniques.

### Example 6

### In vitro Recombination of ET1158, GT6839, or ET5262 Genes by DNA Shuffling

The nucleotide sequence of SEQ ID NO:1, SEQ ID NO:3, or SEQ ID NO:5 is amplified by PCR. The resulting DNA fragment is digested by DNaseI treatment essentially as described (Stemmer et al. (1994) PNAS 91: 10747-10751) and the PCR primers are removed from the reaction mixture. A PCR reaction is carried out without primers and is followed by a PCR reaction with the primers, both as described (Stemmer et al. (1994) PNAS 91: 10747-10751). The resulting DNA fragments are cloned into pTRC99a (Pharmacia, Cat NO:27-5007-01) for use in bacteria, and transformed into a bacterial strain deficient in ET1158, GT6839, or ET5262 activity by electroporation using the Biorad Gene Pulser and the manufacturer's conditions. The transformed bacteria are grown on medium that contains inhibitory concentrations of an inhibitor of ET1158, GT6839, or ET5262 activity, respectively, and those colonies that grow in the presence of the inhibitor are selected. Colonies that grow in the presence of normally inhibitory concentrations of inhibitor are picked and purified by repeated restreaking. Their plasmids are purified and the DNA sequences of cDNA inserts from plasmids that pass this test are then determined. Alternatively, the DNA fragments are cloned into expression vectors for transient or stable transformation into plant cells, which are screened for differential survival and/or growth in the presence of an inhibitor of ET1158, GT6839, or ET5262 activity. In a similar reaction, PCR-amplified DNA fragments comprising the Arabidopsis ET1158, GT6839, or ET5262 gene encoding the protein and PCR-amplified DNA fragments derived from or comprising another ET1158, GT6839, or ET5262 gene are recombined in vitro and resulting variants with improved tolerance to the inhibitor are recovered as described above.

### Example 7

### In vitro Recombination of ET1158, GT6839, or ET5262 Genes by Staggered Extension Process

The Arabidopsis ET1158 gene encoding the protein and another ET1158 gene, or homolog thereof, or fragment thereof, are each cloned into the polylinker of a pBluescript vector. A PCR reaction is carried out essentially as described (Zhao et al. (1998) Nature Biotechnology 16: 258-261) using the “reverse primer” and the “M13-20 primer” (Stratagene Catalog). Amplified PCR fragments are digested with appropriate restriction enzymes and cloned into pTRC99a and mutated ET1158 genes are screened as described in Example 4. The same procedure is carried out with genes encoding GT6839 or ET5262 proteins, respectively.

### Example 8

### In Vitro Binding Assays

Recombinant ET1158 protein is obtained, for example, according to Example 5. The protein is immobilized on chips appropriate for ligand binding assays using techniques which are well known in the art. The protein immobilized on the chip is exposed to sample compound in solution according to methods well know in the art. While the sample compound is in contact with the immobilized protein measurements capable of detecting protein-ligand interactions are conducted. Examples of such measurements are SELDI, biacore and FCS, described above. Compounds found to bind the protein are readily discovered in this fashion and are subjected to further characterization.

Recombinant GT6839 protein is obtained, for example, according to Example 5. The protein is immobilized on chips appropriate for ligand binding assays using techniques which are well known in the art. The protein immobilized on the chip is exposed to sample compound in solution according to methods well know in the art. While the sample compound is in contact with the immobilized protein measurements capable of detecting protein-ligand interactions are conducted. Examples of such measurements are SELDI, biacore and FCS, described above. Compounds found to bind the protein are readily discovered in this fashion and are subjected to further characterization.

Recombinant ET5262 protein is obtained, for example, according to Example 5. The protein is immobilized on chips appropriate for ligand binding assays using techniques which are well known in the art. The protein immobilized on the chip is exposed to sample compound in solution according to methods well know in the art. While the sample compound is in contact with the immobilized protein measurements capable of detecting protein-ligand interactions are conducted. Examples of such measurements are SELDI, biacore and FCS, described above. Compounds found to bind the protein are readily discovered in this fashion and are subjected to further characterization.

### Example 9

### Plastid Transformation

Transformation Vectors

For expression of a nucleotide sequence encoding a polypeptide having ET1158, GT6839, or ET6252 activity encoding in plant plastids, plastid transformation vector pPH143 or pPH 145 (WO 97/32011) is used; and this reference is incorporated herein by reference. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

Plastid Transformation

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif.) coated with DNA from plasmids pPH143 and pPH145 essentially as described (Svab, Z. and Maliga, P. (1993) Proc. Natl. Acad. Sci. USA 90, 913-917). Bombarded seedlings are incubated on T medium for two days after which leaves are excised and placed abaxial side up in bright light (350-500 μmol photons/m²/s) on plates of RMOP medium (Svab, Z., Hajdukiewicz, P. and Maliga, P. (1990) Proc. Natl. Acad. Sci. USA 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). Homoplasmic shoots are rooted aseptically on spectinomycin-containing MS/IBA medium (McBride, K. E. et al. (1994) Proc. Natl. Acad. Sci. USA 91, 7301-7305) and transferred to the greenhouse.

The above disclosed embodiments are illustrative. This disclosure of the invention will place one skilled in the art in possession of many variations of the invention. All such obvious and foreseeable variations are intended to be encompassed by the appended claims.

What is claimed is:

1. An isolated DNA molecule comprising a nucleotide sequence encoding an amino acid sequence substantially similar to SEQ ID NO:2 or SEQ ID NO:6.

2. The DNA molecule of claim 1, wherein said nucleotide sequence is substantially similar to SEQ ID NO:1 or SEQ ID NO:5.

3. The DNA molecule according to claim 1, wherein said nucleotide sequence is a plant nucleotide sequence.

4. The DNA molecule of claim 1, wherein the amino acid sequence has ET1158 or ET5262 activity.

5. A polypeptide comprising an amino acid sequence encoded by a nucleotide sequence identical or substantially similar to SEQ ID NO: I or SEQ ID NO:5.

6. The polypeptide of claim 5, wherein said amino acid sequence is substantially similar to SEQ ID NO:2 or SEQ ID NO:6.

7. The polypeptide of claim 5, wherein said amino acid sequence has ET1158 or ET5262 activity.

8. A polypeptide comprising an amino acid sequence comprising at least 20 consecutive amino acid residues of the amino acid sequence of SEQ ID NO:2 or SEQ ID NO:6.

9. An expression cassette comprising a promoter operatively linked to a DNA molecule comprising a nucleotide sequence encoding an amino acid sequence substantially similar to SEQ ID NO:2, SEQ ID NO:4 or SEQ ID NO:6.

10. A recombinant vector comprising an expression cassette according to claim 9.

11. A host cell comprising a DNA molecule comprising a nucleotide sequence encoding an amino acid sequence substantially similar to SEQ ID NO:2, SEQ ID NO:4 or SEQ ID NO:6.

12. A host cell according to claim 11, wherein said host cell is selected from the group consisting of an insect cell, a yeast cell, a prokaryotic cell and a plant cell.

13. A plant or seed comprising a plant cell of claim 12.

14. A plant of claim 13, wherein said plant is tolerant to an inhibitor of ET1158, GT6839, or ET5262 activity.

15. A method comprising:
a) combining a polypeptide comprising the amino acid sequence encoded by a DNA molecule comprising a nucleotide sequence encoding an amino acid sequence substantially similar to SEQ ID NO:2, SEQ ID NO:4, or SEQ ID NO:6, or a homolog thereof, and a compound to be tested for the ability to interact with said polypeptide, under conditions conducive to interaction; and
b) selecting a compound identified in step (a) that is capable of interacting with said polypeptide.

16. The method according to claim 15, further comprising:
c) applying a compound selected in step (b) to a plant to test for herbicidal activity; and
d) selecting compounds having herbicidal activity.

17. A compound identifiable by the method of claim 15.

18. A compound having herbicidal activity identifiable by the method of claim 16.

19. A process of identifying an inhibitor of ET1158, GT6839, or ET5262 activity comprising:
a) introducing a DNA molecule comprising a nucleotide sequence encoding an amino acid sequence substantially similar to SEQ ID NO:2, SEQ ID NO:4, or SEQ ID NO:6, and encoding a polypeptide having ET1158, GT6839, or ET5262 activity, or a homolog thereof, into a plant cell, such that said sequence is functionally expressible at levels that are higher than wild-type expression levels;
b) combining said plant cell with a compound to be tested for the ability to inhibit the ET1158, GT6839, or ET5262 activity under conditions conducive to such inhibition;
c) measuring plant cell growth under the conditions of step (b);
d) comparing the growth of said plant cell with the growth of a plant cell having unaltered ET1158, GT6839, or ET5262 activity under identical conditions; and
e) selecting said compound that inhibits plant cell growth in step (d).

20. A compound having herbicidal activity identifiable according to the process of claim 19.

21. A method for recombinantly expressing a protein having ET1158, GT6839, or ET5262 activity comprising:
(a) introducing a nucleotide sequence encoding a protein substantially similar to SEQ ID NO:2, SEQ ID NO:4, or SEQ ID NO:6 into a host cell; and
(b) expressing said nucleotide sequence in said host cell.


SEQUENCE LISTING (21 sequences)

SEQ ID NO: 1 - DNA, 1818 bp, Arabidopsis thaliana
  CDS (1)..(1818)
atggctgcttcttcatcctccgctgcttccttctttggagtccgacaagatgaccaatctcacctcctcccacctaattcctccgccgctgctcctcctcctccacctcctcaccaccaggcaccgctgccaccgcttgaagctccaccgcagaaaaagaagagaaaccaaccaagaactccaaattccgatgcggaagtgatagctttatctccaaagacactaatggctacaaacagattcatatgtgaagtatgcaacaaagggtttcaaagagaacagaatctacaacttcaccgaagaggacacaatcttccatggaagctcaaacagaaatcgaccaaagaagtgaagagaaaagtgtatctttgtccggagccctcgtgcgtccaccatgacccgtcacgtgctctcggagacctcaccggaatcaagaaacattattaccgtaaacacggtgaaaagaagtggaaatgcgataaatgctctaagcgttacgctgttcaatcggattggaaagctcactccaagacttgtggtaccaaagagtatcgttgtgactgtggtacactcttctctcggcgagacagtttcatcacacatagagctttctgtgacgcgttggctcaagagagtgcgagacacccaacttcattgacttctttgccaagtcatcacttcccgtacggacaaaacaccaacaactccaacaacaacacttcaagcatgatccttggtctgtcccacatggggcccccacagaatcttgatcaccagtccggtgacgttctccgtcttggaagcggaggaggaggaggaggagccgcttcacgctcttcttctgatctcattgctgcgaatgcttcaggctacttcatgcaagagcaaaaccctagctttcatgatcaacaagatcatcatcatcatcatcatcatcatcaacaagggtttttggctgggaacaataacatcaagcaatcaccaatgagttttcaacagaatctgatgcagttctcacatgataaccataattctgctccctccaatgtcttcaatctcagcttcctctccggaaacaacggagttacttctgccacaagtaaccctaatgctgccgccgctgctgctgtttcttctggtaatcttatgatatcaaaccattatgatggcgaaaatgctgttggaggaggaggagaaggaagcactggtctcttccctaacaatctgatgagctcggcagatagaattagctcaggatcagtcccttcactctttagctcatcaatgcaaagtccaaattcagcacctcacatgtcagccactgcccttctacagaaagctgctcaaatgggttcaacctcaagcaacaacaacaacggaagcaacaccaacaacaataacaatgcctcatcgatcctaagaagctttgggagtggaatctacggagaaaatgagagtaatcttcaggatttgatgaattctttctctaaccccggcgcaacgggaaacgttaacggagttgattctccttttggttcgtacggaggagtgaacaaaggattaagcgctgacaaacagagcatgactagagactttcttggagttggacagatcgtaaaaagcatgagtggaagcggagggtttcaacaacagcaacaacagcaacagcagcaacaacaacaacaacaacatggaaatagtagagaaagagttggctcgtcgtcggattccgctgatagaagcagcatgaatgtgaataccggaggtggtccggcaagtacttcaccaccttatggaatccatcatgcgagtttctaa

SEQ ID NO: 2 - protein, 605 aa, Arabidopsis thaliana
MAASSSSAASFFGVRQDDQSHLLPPNSSAAAPPPPPPHHQAPLPPLEAPPQKKKRNQPRTPNSDAEVIALSPKTLMATNRFICEVCNKGFQREQNLQLHRRGHNLPWKLKQKSTKEVKRKVYLCPEPSCVHHDPSRALGDLTGIKKHYYRKHGEKKWKCDKCSKRYAVQSDWKAHSKTCGTKEYRCDCGTLFSRRDSFITHRAFCDALAQESARHPTSLTSLPSHHFPYGQNTNNSNNNTSSMILGLSHMGPPQNLDHQSGDVLRLGSGGGGGGAASRSSSDLIAANASGYFMQEQNPSFHDQQDHHHHHHHHQQGFLAGNNNIKQSPMSFQQNLMQFSHDNHNSAPSNVFNLSFLSGNNGVTSATSNPNAAAAAAVSSGNLMISNHYDGENAVGGGGEGSTGLFPNNLMSSADRISSGSVPSLFSSSMQSPNSAPHMSATALLQKAAQMGSTSSNNNNGSNTNNNNNASSILRSFGSGIYGENESNLQDLMNSFSNPGATGNVNGVDSPFGSYGGVNKGLSADKQSMTRDFLGVGQIVKSMSGSGGFQQQQQQQQQQQQQQQHGNSRERVGSSSDSADRSSMNVNTGGGPASTSPPYGIHHASF

SEQ ID NO: 3 - DNA, 1023 bp, Arabidopsis thaliana
  CDS (1)..(1023)
atgagcagcacaagcactagttctgctcttatccaccatttccgcctcaccactcgcaacttgggttcgcctacaaagcagcgttgtccttacgctgtaactttctgcaattcgtggagggaagctggactccgatactccgtcacgcaacgccggagcaaaggctttgggccggtgtcagctctcaatgacgacgattcaccaacagagaccacccccggcgttggctctgctgtagaagacagaccaccagattcatctgaagatagatcaagttcggtctatgagtttctgtatccccgtaaagaggagctccctgatgacaaagaaatgactattttcgaccatctcgaggagctccgggagagaatattcgtctctgttttggctgtaggagctgcaatcttgggatgcttcgccttctccaaagatcttattgtctttcttgaagctcccgtcaaaactcagggtgtacggtttctccagcttgctcctggcgaatttttcttcacaactttaaaggtctccggttattgcgggcttctactagggagcccagtgatcctgtatgagattatagcttttgtccttcccggtctgacacgggctgagagaaggtttctggggccaattgtatttggctcctcattgctcttctatgctggacttgctttctcctactgggttttaacccctgcagccttgaatttctttgtgaattacgctgaaggtgtggtagaatctctgtggtctatcgaccagtactttgagtttgtactagtgcttatgttcagcacaggcctttctttccaggttccagtcattcagttactcctgggacaagtaggggttgtgtcgggagatcaaatgctatcaatttggagatatgtggtggtaggtgcggtggttgcagcagctgttgtcacaccctcgacagaccctgtcactcaaatgctcctagcaacaccgcttctggggctctacttgggtggtgcatggatggtcaagctcacaggtcggtga

SEQ ID NO: 4 - protein, 340 aa, Arabidopsis thaliana
MSSTSTSSALIHHFRLTTRNLGSPTKQRCPYAVTFCNSWREAGLRYSVTQRRSKGFGPVSALNDDDSPTETTPGVGSAVEDRPPDSSEDRSSSVYEFLYPRKEELPDDKEMTIFDHLEELRERIFVSVLAVGAAILGCFAFSKDLIVFLEAPVKTQGVRFLQLAPGEFFFTTLKVSGYCGLLLGSPVILYEIIAFVLPGLTRAERRFLGPIVFGSSLLFYAGLAFSYWVLTPAALNFFVNYAEGVVESLWSIDQYFEFVLVLMFSTGLSFQVPVIQLLLGQVGVVSGDQMLSIWRYVVVGAVVAAAVVTPSTDPVTQMLLATPLLGLYLGGAWMVKLTGR

SEQ ID NO: 5 - DNA, 555 bp, Arabidopsis thaliana
  CDS (1)..(555)
atggcagtttttgttcttgtgatgattttgttggccatggctggtcactcaagtgggacatggtgtgtatgcaaagaagggttaagcgaggcaatgctgcagaagacattggactacgcatgtggtgcaggagctgattgtggacccattcaccagaccgggccttgctttaaccctaacaccgtcaagtctcactgctcctacgccgtcaacagcttctttcagaagaaaggtcagtctctgggcacttgtgactttgctggcacagccaccttctcagcctctgatcccagctacactacttgtcctttccctgcaagtgccagtggaagtgggacaacgactccagtgacgacaacaccttcgacaagagtccctacaacaactaatacaagaccctacacaatcacaccatcaacaggaggaggtttgggaataccgtctggtattaacccggattacacagatccatcctttggattcaaactccaaagcccaagatttggattcatagtcttgttcactctcttcctacccttttacttgtttagctaa

SEQ ID NO: 6 - protein, 184 aa, Arabidopsis thaliana
MAVFVLVMILLAMAGHSSGTWCVCKEGLSEAMLQKTLDYACGAGADCGPIHQTGPCFNPNTVKSHCSYAVNSFFQKKGQSLGTCDFAGTATFSASDPSYTTCPFPASASGSGTTTPVTTTPSTRVPTTTNTRPYTITPSTGGGLGIPSGINPDYTDPSFGFKLQSPRFGFIVLFTLFLPFYLFS

SEQ ID NO: 7 - DNA, 18 bp, Artificial Sequence
  Description of Artificial Sequence oligonucleotide
ngttgwgnatwtsgwgnt

SEQ ID NO: 8 - DNA, 15 bp, Artificial Sequence
  Description of Artificial Sequence oligonucleotide
tgwgnagsancasag

SEQ ID NO: 9 - DNA, 16 bp, Artificial Sequence
  Description of Artificial Sequence oligonucleotide
agwgnagwancawagg

SEQ ID NO: 10 - DNA, 16 bp, Artificial Sequence
  Description of Artificial Sequence oligonucleotide
sttgntastnctntgc

SEQ ID NO: 11 - DNA, 15 bp, Artificial Sequence
  Description of Artificial Sequence oligonucleotide
ntcgastwtsgwgtt

SEQ ID NO: 12 - DNA, 16 bp, Artificial Sequence
  Description of Artificial Sequence oligonucleotide
wgtgnagwancanaga

SEQ ID NO: 13 - DNA, 30 bp, Artificial Sequence
  Description of Artificial Sequence oligonucleotide
actagctctaccgtttccgtttccgtttac

SEQ ID NO: 14 - DNA, 30 bp, Artificial Sequence
  Description of Artificial Sequence oligonucleotide
ttacctcgggttcgaaatcgatcgggataa

SEQ ID NO: 15 - DNA, 33 bp, Artificial Sequence
  Description of Artificial Sequence oligonucleotide
aaaatcggttatacgataacggtcggtacggga

SEQ ID NO: 16 - DNA, 36 bp, Artificial Sequence
  Description of Artificial Sequence oligonucleotide
gggtcttgcggatctgaatatatgttttcatgtgtg

SEQ ID NO: 17 - DNA, 37 bp, Artificial Sequence
  Description of Artificial Sequence oligonucleotide
taccgaagaaaaataccggttcccgtccgatttcgac

SEQ ID NO: 18 - DNA, 34 bp, Artificial Sequence
  Description of Artificial Sequence oligonucleotide
ggatcgtatcggttttcgattaccgtatttatcc

SEQ ID NO: 19 - DNA, 3636 bp, Arabidopsis thaliana
ttagaaactcgcatgatggattccataaggtggtgaagtacttgccggaccacctccggtattcacattcatgctgcttctatcagcggaatccgacgacgagccaactctttctctactatttccatgttgttgttgttgttgttgctgctgttgctgttgttgctgttgttgaaaccctccgcttccactcatgctttttacgatctgtccaactccaagaaagtctctagtcatgctctgtttgtcagcgcttaatcctttgttcactcctccgtacgaaccaaaaggagaatcaactccgttaacgtttcccgttgcgccggggttagagaaagaattcatcaaatcctgaagattactctcattttctccgtagattccactcccaaagcttcttaggatcgatgaggcattgttattgttgttggtgttgcttccgttgttgttgttgcttgaggttgaacccatttgagcagctttctgtagaagggcagtggctgacatgtgaggtgctgaatttggactttgcattgatgagctaaagagtgaagggactgatcctgagctaattctatctgccgagctcatcagattgttagggaagagaccagtgcttccttctcctcctcctccaacagcattttcgccatcataatggtttgatatcataagattaccagaagaaacagcagcagcggcggcagcattagggttacttgtggcagaagtaactccgttgtttccggagaggaagctgagattgaagacattggagggagcagaattatggttatcatgtgagaactgcatcagattctgttgaaaactcattggtgattgcttgatgttattgttcccagccaaaaacccttgttgatgatgatgatgatgatcttgttgatcatgaaagctagggttttgctcttgcatgaagtagcctgaagcattcgcagcaatgagatcagaagaagagcgtgaagcggctcctcctcctcctcctccgcttccaagacggagaacgtcaccgggctggtgatcaagattctgtggggcccccatgtgggacagaccaaggatcatgcttgaagcgttgttgttggagttgtttgtgttttgtccgtacgggaagtgatgacttggcaaagaagtcaatgaagttgggtgtctcgcactctcttgagccaacgcgtcacagaaagctctatgtgtgatgaaactgtctcgcctgtgacacaaaataaaataaataaaaatcaatttcacaatggaatctattggatcgtcagatatatctctatactattcatataaaatgatgaaataaaagacatattttgggaaaagtgaaagaaaaaaaatataaatgtaagtagagagagagactcatcatgtcacgtagctacaaatagattcgcgtgtaattatgggatcttgaaaagtagatagatggctagaaagtcactctactactctattatacatcatattattattcacctgtgtaaactcttaacaagcttacacactcacatgtttgatgcatacaataaaattgcacgcatacacataaataggagtacaatgttgtgtcacatttcaactatgatgatgtatttgtctatatgactatgtgtacgaacaaatctaactcttatcaatatcagtttatgcaataataataaacataaacattattaatcaactccggaaaactattatgcttaattcactataatttatgtatgaaactatgaatatagctatctatataataaattaatgtggtattcaacgaattccaaaggtaggtggaaaaagttaagtttcacactaatgtatagagaatgattgtcaaatgaatatctgagtttgtcctaattcaagatcttgtatgttcaataaatgcaagttgagcatgtattcaatgaactacttatatttttatttaagaaaactagctacttatatatatttgcagcaccacatatttatttacttcattctatttatagaactcctctaaatgcacgcacacacacaaacacagcaatgccagccaagactgagaaaaaaactctatattcgaaataattatttagtctattagttatcacgtctatgcaaatttatgccattttatgcaaaatgtgtagagtagtgaaccaaactaatatgtggctatggtacagagtacaaacgaatcatatgaaataattgtattttcgtaaaattttacacataagagaacaaacactgtaacaacaacctccctagatattatatagtttagcagcacgtgaattgcccatatagtataagtataatttcatatccaagaaaaaaaaatcctgagaaaaatatattaatttatttttatgacagaaaatagggcggtgtttccaaggcaaaagaatatttaaaaaaaaaactgaaattgaaattggtcctctttggttttgagtttctgctagaaagcatctattgaaatatggcagtttcagtgtcatctatagatatagttatatagacacacacacacattcacgtgtgttcttgtatcatttttttttataaaaaaaaaaagaaaaaaaaaactaaagttgcatttactgcacgaaagaaagcgataagagcatcgaagacttcgaggcgaaacagaaagtgaacctaaaaaaaaccctaattcatgaagaatgactcacagacacatattcacacacatatcactatagcagctcaatagattaaattataatttaaagagtttgaaagatttggattactaaccgagagaagagtgtaccacagtcacaacgatactctttggtaccacaagtcttggagtgagctttccaatccgattgaacagcgtaacgcttagagcatttatcgcatttccacttcttttcaccgtgtttacggtaataatgtttcttgattccggtgaggtctccgagagcacgtgacgggtcatggtggacgcacgagggctccggacaaagatacacttttctcttcacttctttggtcgatttctgtttgagcttccatggaagattgtgtcctcttcggtgaagttgtagattctgttctctttgaaaccctttgttgcatacttcacatatgaatctgtttgtagccattagtgtctttggagataaagctatcacttccgcatcggaatctaatcaagaggaaaattattaactaaattttaaaagtcttatattgcatggaagataaattgattgaaatctgaattttgaaaaaccaactacaagctagggtttgctaatttgcttatttcaagattttaaaaaatgtaaaagggagaagaaaaaaaaggacatggaaaagggatttatctttttcaccaatcaataagttcttttaagaaatgatcacatgattataaaagatttgaattaagaggtaattaacttacttggagttcttggttggtttctcttctttttctgcggtggagcttcaagcggtggcagcggtgcctggtggtgaggaggtggaggaggaggagcagcggcggaggaattaggtgggaggaggtgagattggtcatcttgtcggactccaaagaaggaagcagcggaggatgaagaagcagccatggat

SEQ ID NO: 20 - DNA, 1303 bp, Arabidopsis thaliana
atgagcagcacaagcactagttctgctcttatccaccatttccgcctcaccactcgcaacttgggttcgcctacaaagcagcgttgtccttacgctgtaactttctgcaattcgtggagggaagctggactccgatactccgtcacgcaacgccggagcaaaggctttgggccggtgtcagctctcaatgacgacgattcaccaacagagaccacccccggcgttggctctgctgtagaagacagaccaccaggtatttattattaggagaaactccactttcttgattgatttttattatcatctccccgggttgttgatatagttagttagttagttagtgatgaattacacactgctgctgtggcagattcatctgaagatagatcaagttcggtctatgagtttctgtatccccgtaaagaggagctccctgatgacaaagaaatgactattttcgaccatctcgaggagctccgggagagaatattcgtctctgttttggctgtaggagctgcaatcttgggatgcttcgccttctccaaagatcttattgtctttcttgaagctcccgtcaaaactcagggtgtacggtttctccagcttgctcctggcgaatttttcttcacaactttaaaggtaatgatcagattgacgacagatagcatcatattcaagatagatgattttctttttcttgtgttgcggtgcaggtctccggttattgcgggcttctactagggagcccagtgatcctgtatgagattatagcttttgtccttcccggtctgacacgggctgagagaaggtttctggggccaattgtatttggctcctcattgctcttctatgctggacttgctttctcctactgggttttaacccctgcagccttgaatttctttgtgaattacgctgaaggtgtggtagaatctctgtggtctatcgaccagtactttgagtttgtactagtgcttatgttcagcacaggcctttctttccaggtatgtggcaatatgatattatacatgttcctaaacgttgctttagccatcttcacctggaattttggctgcttcttgttggtttacaggttccagtcattcagttactcctgggacaagtaggggttgtgtcgggagatcaaatgctatcaatttggagatatgtggtggtaggtgcggtggttgcagcagctgttgtcacaccctcgacagaccctgtcactcaaatgctcctagcaacaccgcttctggggctctacttgggtggtgcatggatggtcaagctcacaggtcggtga

SEQ ID NO: 21 - DNA, 1449 bp, Arabidopsis thaliana
  misc_feature (1)..(1449): n=a, t, g, or c.
gactctctttctctctctttattcacacctcttgttaccttcaaaaccttctctcaatctcctccattatccttctctttttctctctccntgcaactggctccatcccaaagtgcttcgttctggtacagcgaccatggcagtttttgttcttgtgatgattttgttggccatggctggtcactcaagtaagtatatcttcgtctcttttywartmsttctstgyctcatatagatcctcttcttttcttctatggcaaaacgattcttcttttctttgttcacccactctgttttacaacagaacaacatagtcacaaacttcttagctttcatagtttttaatgaaattttccacaaagaagggccatttttgcttttacccataccttcttttcatatttcctttttgactgtggagttcaacaatcttgtcccctcttgaattaaacattaaggtgttccatttcacacattatcagtgaatgagatttgaagaatgctcattgaagtgtttatgtgtgtctttaggtgggacatggtgtgtatgcaaagaagggttaagcgaggcaatgctgcagaagacattggactacgcatgtggtgcaggagctgattgtggacccattcaccagaccgggccttgctttaaccctaacaccgtcaagtctcactgctcctacgccgtcaacagcttctttcagaagaaaggtcagtctctgggcacttgtgactttgctggcacagccaccttctcagcctctgatcccagtgagtctctcctaccaacctcactacattagaattagtgcaaaaacaatttgttcatatttggtaacttaactaggtaaagttaaaagcagtagttgaaaaatggaacccaacttgaaagcaaatcagaaaggctagtttatattaatctaaacttcacatgtttcaatgtgaatgatatgataaataaagtatccattatcttattatatagtatcacaactcaagattctctacacagatttgaaactagattgtagaacatatcatttttattagtaactgttgtctcttgtaccattgtggtcttatgatataggctacactacttgtcctttccctgcaagtgccaggtacacacccttctttctttcttcacttcttatttgttgttttgcaaacaagaattggctttggtcttgaggatctttgcactctagatctttgtttaagctgattgatacaatctgagagatgtgtttgtatgtttgttgttatttaccatttaatgcaggggaagtgggacaacgactccagtgacgacaacaccttcgacaagagtccctacaacaactaatacaagaccctacacaatcacaccatcaacaggaggaggtttgggaataccgtctggtattaacccggattacacagatccatcctttgctactacactaa